SEQUENCE LISTING (2 sequences)

SEQ ID NO: 1 - DNA, 5180 bp, Microorganism
  CDS (877)...(4731)
atctaccggtttttgtgaagtttggcagtattcttccgatgaatttgaacgcgcaatatcaagtgggcgggaccattggcaacagcttgacgagctacacgaatctcgcgttccgcatttatccgcttgggacaacaacgtacgactggaatgatgatattggcggttcggtgaaaaccataacttctacagagcaatatgggttgaataaagaaaccgtgactgttccagcgattaattctaccaagacattgcaagtgtttacgactaagccttcctctgtaacggtgggtggttctgtgatgacagagtacagtactttaactgccctaacgggagcgtcgacaggctggtactatgatactgtacagaaattcacttacgtcaagcttggttcaagtgcatctgctcaatccgttgtgctaaatggcgttaataaggtggaatatgaagcagaattcggcgtgcaaagcggcgtttcaacgaacacgaaccatgcaggttatactggtacaggatttgtggacggctttgagactcttggagacaatgttgcttttgatgtttccgtcaaagccgcaggtacttatacgatgaaggttcggtattcatccggtgcaggcaatggctcaagagccatctatgtgaataacaccaaagtgacggaccttgccttgccgcaaacaacaagctgggatacatgggggactgctacgtttagcgtctcgctgagtacaggtctcaacacggtgaaagtcagctatgatggtaccagttcacttggcattaatttcgataacatcgcgattgtagagcaataaaaggtcgggagggcaagtccctcccttaatttctaatcgaaagggagtatccttgatgcgtccaccaaacaaagaaattccacgtattcttgctttttttacagcgtttacgttgtttggttcaacccttgccttgcttcctgctccgcctgcgcatgcctatgtcagcagcctaggaaatctcatttcttcgagtgtcaccggagataccttgacgctaactgttgataacggtgcggagccgagtgatgacctcttgattgttcaagcggtgcaaaacggtattttgaaggtggattatcgtccaaatagcataacgccgagcgcgaagacgccgatgctggatccgaacaaaacttggtcagctgtaggagctacgattaatacgacagccaatccaatgaccatcacgacttccaatatgaagattgagattaccaagaatccagtacgaatgacggtcaagaaggcggacggcactacgctattctgggaaccatcaggcggaggggtattctcagacggtgtgcgcttccttcatgccacaggggataatatgtatggcatccggagcttcaatgcttttgatagcgggggtgacctgctgcggaattcgtccaatcatgccgcccatgcgggtgaacagggagattccggtggtccgcttatttggagtacggcaggatatggactattagtcgatagcgatggcggctacccctatacagatagcacaaccggtcaaatggagttttattatggtgggacccctcctgagggacgtcgttatgcgaaacaaaacgtggaatattatattatgctcggaacccccaaggaaattatgaccgacgtaggggaaatcacagggaaaccgcctatgctgcctaagtggtcgcttggattcatgaactttgagtgggatacgaatcaaacggagtttacgaataatgtggatacgtatcgtgccaaaaatatccccatagatgcttacgccttcgactatgactggaaaaagtacggggaaaccaactatggtgaattcgcgtggaatacgactaatttcccttctgcgtcaacgacttctttaaagtcaacaatggatgctaaaggcatcaaaatgatcggaattacaaaaccccgcatcgttacgaaggatgcttcagcgaatgtgacgacccaagggacggacgcgacaaatggcggttatttttatccaggccataacgagtatcaggattatttcattcccgtaactgtgcgtagtatcgatccttacaatgctaacgaacgtgcttggttctggaatcattccacagatgcgcttaataaagggatcgtaggttggtggaatgacgagacggataaagtatcttcgggtggagcgttatattggtttggcaatttcacaacaggccacatgtctcagacgatgtacgaaggggggcgggcttacacgagtggagcgcagcgtgtttggcaaacggctagaaccttctacccaggtgcccagcggtatgcgactacgctttggtctggcgatattggcattcaatacaataaaggcgaacggatcaattgggctgccgggatgcaggagcaaagggcagttatgctatcctccgtgaacaatggccaggtgaaatggggcatggataccggcggattcaatcagcaggatggcacgacgaacaatccgaatcccgatttatacgctcggtggatgcagttcagtgccctaacgcctgttttccgagtgcatgggaacaaccatcagcagcgccagccatggtacttcggatcgactgcggaggaggcctccaaagaggcaattcagctgcggtactccctgatcccttatatgtatgcctatgagagaagtgcttacgagaatgggaatgggctcgttcggccattgatgcaagcctatccaacagatgcggccgtcaaaaattacacggatgcttggatgtttggtgactggctgctggctgcacctgtggtagataaacagcagacgagtaaggatatctatttaccgtctgggtcatggattgactatgcgcgaggcaatgcaataactggcggtcaaaccatccgatattcggttaatccggacacgttgacagacatgcctctctttattaaaaaaggtgccattattccaacacagaaagtgcaggattacgtagggcaggcttccgtcacttccgttgatgtggatgtgtttccggatacgacgcagtcgagtttcacgtactacgatgatgatggcgccagttataactatgagagcggcacttattttaagcaaaatatgactgctcaggataatgggtcaggctcgttaagttttactttaggagcaaagagtggcagttacacgccggctctccaatcctatatcgttaagctgcacggttctgctggaacttctgttacgaataacagcgcagctatgacatcttatgcaagcttggaagcattaaaagctgctgctggggaaggctgggcgactgggaaggacatttatggggatgtcacctatgtgaaagtgacggcaggtacagcttcttctaaatctattgctgttacaggtgttgctgccgtgagcgcaactacttcgcaatacgaagctgaggatgcatcgctttctggcaattcggttgctgcaaaggcgtccataaacacgaatcataccggatatacgggaactggatttgtagatggtttggggaatgatggcgctggtgtcaccttctatccaaaggtgaaaactggcggtgactacaatgtctccttgcgttatgcgaatgcttcaggcacggctaagtcagtcagtatttttgttaatggaaaaagagtgaagtccacctcgctcgctaatctcgcaaattgggacacttggtctacacaatctgagacactgccgttgacggcaggtgtgaatgttgtgacctataaatattactccgatgcgggagatacaggcaatgttaacatcgacaacatcacggtaccttttgcgccaattatcggtaagtatgaagcagagagtgctgagctttctggtggcagctcattgaacacgaaccattggtactacagtggtacggcttttgtagacggtttgagtgctgtaggcgcgcaggtgaaatacaacgtgaatgtccctagcgcaggaagttatcaggtagcgctgcgatatgcgaatggcagtgcagcgacgaaaacgttgagtacttatatcaatggagccaagctggggcaaaccagttttacgagtcctggtacgaattggaatgtttggcaggataatgtgcaaacggtgacgttaaatgcaggggcaaacacgattgcgtttaaatacgacgccgctgacagcgggaacatcaacgtagatcgtctgcttctttcaacttcggcagcgggaacgccggtttctgagcagaacctgctagacaatcccggtttcgagcgtgacacgagtcaaaccaataactggattgagtggcatccaggcacgcaagctgttgcttttggcgttgatagcggctcaaccaccaatccgccggaatccccgtggtcgggtgataagcgtgcctacttctttgcagcaggtgcctatcaacaaagcatccatcaaaccattagtgttcctgttaataatgtaaaatacaaatttgaagcctgggtccgcatgaagaatacgacgccgacgacggcaagagccgaaattcaaaactatggcggatcagccatttatgcgaacataagtaacagcggtgtttggaaatatatcagcgtaagtgatattatggtgaccaatggtcagatagatgttggattttacgtggattcacctggtggaactacgcttcacattgatgatgtgcgcgtaaccaaacaataaacaaacaaccagctctcccgttaatgggagggctggttgtttgttatgataatccatctatttagagtggattaaacgttttgaagtgcttgctgaacttcttgcacaatggataacgccgcggtgcgggcacttgagaaagcacgttctgcaagctctcccttacctgtacagccgtctccgcagaagtagaaaggaacgttttccacgcgtatcggcagcagattattggaagcaatgtttttcacgctggaaaccatcgctttcttggaaacccgtttcacggctgtgacatcgcgccagcctggataatgtttatcaaataaggcttccatttggaggttcttctcttccaggtacgctttgcgctgctcctcgttatcaaagcggtcgcttaagtatgcgataccttgcagcagctgcccgccttctggtactagtgtgtgatc

SEQ ID NO: 2 - DNA, 3869 bp, Microorganism
  CDS (241)...(3522)
tcatcgctactggcaatcggattcaaacaaatggctgcagctcgcacagacgattgtggaaagggaatatctgatttaaccatacggcggtcgcgattgattgaataggattcgtggccgcctaatattgaaaggggggatgcgtggagcagcgcatgcacggcgaggaataactgttgttggagcctctaagtcattcatgtttagcaaacaaatttcggtacgaaaggggaaatgtttatgtatgtaaggaatctaacaggttcattccgattttctctctcttttttgctctgtttctgtctcttcgtcccctctatttatgccattgatggtgtttatcatgcgccatacggaatcgatgatctgtacgagattcaggcgacggagcggagtccaagagatcccgttgcaggcgatactgtgtatatcaagataacaacgtggcccattgaatcaggacaaacggcttgggtgacctggacgaaaaacggtgtcaatcaagctgctgtcggagcagcattcaaatacaacagcggcaacaacacttactgggaagcgaaccttggcacttttgcaaaaggggacgtgatcagttataccgttcatggcaacaaggatggcgcgaatgagaaggttatcggtccttttacttttaccgtaacgggatgggaatccgttagcagtatcagctctattacggataatacgaaccgtgttgtgctgaatgcggtgccgaatacaggcacattgaagccaaagatcaacctttcctttacggcggatgatgtcctccgcgtacaggtttctccaaccggaacaggaacgttaagcagtggacttagtaattacacagtttcagataccgcctcaaccacttggcttacaacttccaagctgaaggtgaaggtggataagaatccattcaaacttagtgtgtataagcctgatggaacgacgttgattgcccgtcaatatgacagcactacgaatcgtaacattgcctggttaaccaatggcagtacaatcatcgacaaggtagaagatcatttttattcaccggcttccgaggagttttttggctttggagagcattacaacaacttccgtaaacgcggaaatgatgtggacacctatgtgttcaaccagtataagaatcaaaatgaccgcacctacatggcaattccttttatgcttaacagcagcggttatggcattttcgtaaattcaacgtattattccaaatttcggttggcaaccgaacgcaccgatatgttcagctttacggctgatacagggggtagtgccgcctcgatgctggattattatttcatttacggtaatgatttgaaaaatgtggtgagtaactacgctaacattaccggtaagccaacagcgctgccgaaatgggctttcgggttatggatgtcagctaacgagtgggatcgtcaaaccaaggtgaatacagccattaataacgcgaactccaataatattccggctacagcggttgtgctcgaacagtggagtgatgagaacacgttttatattttcaatgatgccacctataccccgaaaacgggcagtgctgcgcatgcctataccgatttcactttcccgacatctgggagatggacggatccaaaagcgatggcagacaatgtgcataacaatgggatgaagctggtgctttggcaggtccctattcagaaatggacttcaacgccctatacccagaaagataatgatgaagcctatatgacggctcagaattatgcagttggcaacggtagcggaggccagtacaggataccttcaggacaatggttcgagaacagtttgctgcttgattttacgaatacggccgccaaaaactggtggatgtctaaacgcgcttatctgtttgatggtgtgggtatcgacggcttcaaaacagatggcggtgaaatggtatggggtcgctcaaatactttctcaaacggtaagaaaggcaatgaaatgcgcaatcaatacccgaatgagtatgtgaaagcctataacgagtacgcgcgctcgaagaaagccgatgcggtctcctttagccgttccggcacgcaaggcgcacaggcgaatcagattttctggtccggtgaccaagagtcgacgtttggtgcttttcaacaagctgtgaatgcagggcttacggcaagtatgtctggcgttccttattggagctgggatatggcaggctttacaggcacttatccaacggctgagttgtacaaacgtgctactgaaatggctgcttttgcaccggtcatgcagtttcattccgagtctaacggcagctctggtatcaacgaggaacgttctccatggaacgcacaagcgcgtacaggcgacaatacgatcattagtcattttgccaaatatacgaatacgcgcatgaatttgcttccttatatttatagcgaagcgaagatggctagtgatactggcgttcccatgatgcgcgccatggcgcttgaatatccgaaggacacgaacacgtacggtttgacacaacagtatatgttcggaggtaatttacttattgctcctgttatgaatcagggagaaacaaacaagagtatttatcttccgcagggggattggatcgatttctggttcggtgctcagcgtcctggcggtcgaacaatcagctacacggccggcatcgatgatctaccggtttttgtgaagtttggcagtattcttccgatgaatttgaacgcgcaatatcaagtgggcgggaccattggcaacagcttgacgagctacacgaatctcgcgttccgcatttatccgcttgggacaacaacgtacgactggaatgatgatattggcggttcggtgaaaaccataacttctacagagcaatatgggttgaataaagaaaccgtgactgttccagcgattaattctaccaagacattgcaagtgtttacgactaagccttcctctgtaacggtgggtggttctgtgatgacagagtacagtactttaactgccctaacgggagcgtcgacaggctggtactatgatactgtacagaaattcacttacgtcaagcttggttcaagtgcatctgctcaatccgttgtgctaaatggcgttaataaggtggaatatgaagcagaattcggcgtgcaaagcggcgtttcaacgaacacgaaccatgcaggttatactggtacaggatttgtggacggctttgagactcttggagacaatgttgcttttgatgtttccgtcaaagccgcaggtacttatacgatgaaggttcggtattcatccggtgcaggcaatggctcaagagccatctatgtgaataacaccaaagtgacggaccttgccttgccgcaaacaacaagctgggatacatgggggactgctacgtttagcgtctcgctgagtacaggtctcaacacggtgaaagtcagctatgatggtaccagttcacttggcattaatttcgataacatcgcgattgtagagcaataaaaggtcgggagggcaagtccctcccttaatttctaatcgaaagggagtatccttgatgcgtccaccaaacaaagaaattccacgtattcttgctttttttacagcgtttacgttgtttggttcaacccttgccttgcttcctgctccgcctgcgcatgcctatgtcagcagcctaggggaaaatctcatttcttcgagtgtcaccggagataccttgacgctaactgttgataacggtgcgccgagtgatgacctcttgattgttcaagcggtgcaaaacggtattttgaaggtggattatcgtccaaatagcataacgccgagcgcgaagacgccgatgctggatc

(19) United States
(12) Patent Application Publication
(10) Pub. No.: US 2004/0253690 A1
(43) Pub. Date: Dec. 16, 2004

(54) Process for producing isomaltose and use thereof

(76) Inventors: Michio Kubota, Okayama (KP); Tomoyuki Nishimoto, Okayama (JP); Takanobu Higashiyama, Okayama (JP); Hikaru Watanabe, Okayama (JP); Shigeharu Fukuda, Okayama (JP); Toshio Miyake, Okayama (JP)

Correspondence Address: Browdy & Neimark, 624 Ninth Street NW, Washington, DC, 20001-5303, US

(21) Appl. No.: 10/363,556

(22) PCT Filed: Apr. 25, 2002
(86) PCT No.: PCT/JP02/04166

(30) Foreign Application Priority Data
Apr. 27, 2001 (JP) 2001-130922

(51) Int. Cl. C12P 19/12

(52) U.S. Cl. 435/100

(57) ABSTRACT

The object of the present invention is to provide a novel process for producing isomaltose and uses thereof and is solved by providing a process for producing isomaltose characterized in that it comprises the steps of allowing α-isomaltosylglucosaccharide-forming enzyme, in the presence or the absence of α-isomaltosyl-transferring enzyme, to act on saccharides, which have a glucose polymerization degree of at least two and α-1,4 glucosidic linkage as a linkage at the non-reducing end, to form α-isomaltosylglucosaccharides, which have a glucose polymerization degree of at least three, α-1,6 glucosidic linkage as a linkage at the non-reducing end, and α-1,4 glucosidic linkage as a linkage other than the non-reducing end, and/or to form cyclo{→6)-α-D-glucopyranosyl-(1→3)-α-D-glucopyranosyl-(1→6)-α-D-glucopyranosyl-(1→3)-α-D-glucopyranosyl-(1→}; allowing isomaltose-releasing enzyme to act on the formed saccharides to release isomaltose; and collecting the released isomaltose; and uses thereof.

### TECHNICAL FIELD

The present invention relates to a novel process for producing isomaltose and uses thereof, more particularly, a process for producing isomaltose from saccharides, which have both a glucose polymerization degree of at least two and an α-1,4 glucosidic linkage as a linkage at the non-reducing end, in a relatively high yield.

### BACKGROUND ART

Isomaltose is a substantially non-crystallizable saccharide which is slightly present in fermented foods and has a relatively low sweetness and satisfactory humectancy. The saccharide has been widely used in a mixture form with saccharides such as glucose, maltose and panose in foods, cosmetics, pharmaceuticals, etc.

Isomaltose is a rare saccharide slightly present in fermented foods, etc., in the natural world. On an industrial-scale production, the following methods for producing isomaltose have been known; partial hydrolysis reaction of dextrans using acid catalysts, enzymatic reactions using dextranase or isomaltodextranase, etc., reverse synthetic reactions from glucose using glucoamylase or acid catalysts, and glucose saccharide-transferring reactions from maltose or maltodextrins using α-glucosidase. However, the isomaltose content of reaction mixtures obtained by conventional methods is only about 10 to about 25% (w/w), on a dry solid basis (d.s.b.) (throughout the specification, “% (w/w)” is abbreviated as “%”, unless specified otherwise), and therefore it is far from satisfaction in view of the purity of isomaltose on an industrial-scale production. As a method for improving the drawback, a column chromatography, as disclosed in Japanese Patent Kokai No. 72,598/83, can be exemplified. According to the method, a high purity isomaltose is obtained from a material saccharide solution with an isomaltose content of about 10 to about 25%, d.s.b. However, the method has the drawback that the purity and yield of isomaltose inevitably depends on the isomaltose content in the material saccharide solutions used.

Under the background, it has been in a great demand a novel process for producing isomaltose on an industrial scale, at a lesser cost, and in a relatively high yield.

In view of the prior arts, the object of the present invention is to establish a process for producing isomaltose which produces isomaltose on an industrial scale, at a lesser cost, and in a relatively high yield.

### DISCLOSURE OF INVENTION

During the present inventors' energetic studying to solve the above object, it has reported in European Journal of Biochemistry, Vol. 226, pp. 641-648 (1994) a cyclic tetrasaccharide having the structure of cyclo{→6)-α-D-glucopyranosyl-(1→3)-α-D-glucopyranosyl-(1→6)-α-D-glucopyranosyl-(1→3)-α-D-glucopyranosyl-(1→} (abbreviated as “cyclotetrasaccharide” throughout the specification), formed by allowing a hydrolyzing enzyme, i.e., alternanase, to act on alternan linked with glucose residues via the alternating α-1,3 and α-1,6 linkages.

While, in the specification of Japanese Patent Application No. 229,557/00, the present inventors disclosed a process for producing cyclotetrasaccharide using an α-isomaltosyl-transferring enzyme which forms cyclotetrasaccharide from saccharides such as panose derived from starches; and disclosed in Japanese Patent Application No. 234,937/00 a process for producing cyclotetrasaccharide in a satisfactorily high yield by allowing the above α-isomaltosyl-transferring enzyme and an α-isomaltosylglucosaccharide-forming enzyme which forms α-isomaltosylglucosaccharide from maltooligosaccharides.

Thereafter, the present inventors focused on the fact that the above α-isomaltosylglucosaccharide and cyclotetrasaccharide have an isomaltose structure intramolecularly, and then studied a method for producing isomaltose from these saccharides. As the result of studying on the enzymatic reaction mechanisms of the above α-isomaltosylglucosaccharide-forming enzyme and α-isomaltosyl-transferring enzyme, the present inventors found that the production yield of isomaltose is outstandingly improved by allowing α-isomaltosylglucosaccharide-forming enzyme and isomaltose-releasing enzyme capable of releasing isomaltose, in the presence or the absence of α-isomaltosyl-transferring enzyme, to act on saccharides having both a glucose polymerization degree of at least two and α-1,4 glucosidic linkage as a linkage at the non-reducing end; and found that the method is easily feasible on an industrial scale. The present inventors also established the uses of isomaltose thus obtained, and accomplished this invention: They accomplished the following process and uses thereof and solved the object of the present invention; a process for producing isomaltose characterized in that it comprises the steps of allowing α-isomaltosylglucosaccharide-forming enzyme, in the presence or the absence of α-isomaltosyl-transferring enzyme, to act on saccharides, which have both a glucose polymerization degree of at least two and α-1,4 glucosidic linkage as a linkage at the non-reducing end, to form α-isomaltosylglucosaccharides, which have a glucose polymerization degree of at least three, α-1,6 glucosidic linkage as a linkage at the non-reducing end, and α-1,4 glucosidic linkage as a linkage other than the non-reducing end, and/or to form cyclo{→6)-α-D-glucopyranosyl-(1→3)-α-D-glucopyranosyl-(1→6)-α-D-glucopyranosyl-(1→3)-α-D-glucopyranosyl-(1→}; allowing isomaltose-releasing enzyme to act on the formed saccharide(s) to release isomaltose; and collecting the released isomaltose.

### BRIEF DESCRIPTION OF DRAWINGS

FIG. 1 shows the thermal influence on the enzymatic activity of α-isomaltosylglucosaccharide-forming enzyme from a microorganism of Bacillus globiformis C9 strain.

FIG. 2 shows the pH influence on the enzymatic activity of α-isomaltosylglucosaccharide-forming enzyme from a microorganism of Bacillus globiformis C9 strain.

FIG. 3 shows the thermal stability of α-isomaltosylglucosaccharide-forming enzyme from a microorganism of Bacillus globiformis C9 strain.

FIG. 4 shows the pH stability of α-isomaltosylglucosaccharide-forming enzyme from a microorganism of Bacillus globiformis C9 strain.

FIG. 5 shows the thermal influence on the enzymatic activity of α-isomaltosyl-transferring enzyme from a microorganism of Bacillus globiformis C9 strain.

FIG. 6 shows the pH influence on the enzymatic activity of α-isomaltosyl-transferring enzyme from a microorganism of Bacillus globiformis C9 strain.

FIG. 7 shows the thermal stability of α-isomaltosyl-transferring enzyme from a microorganism of Bacillus globiformis C9 strain.

FIG. 8 shows the pH stability of α-isomaltosyl-transferring enzyme from a microorganism of Bacillus globiformis C9 strain.

FIG. 9 shows the thermal influence on the enzymatic activity of α-isomaltosylglucosaccharide-forming enzyme from a microorganism of Bacillus globisporus C11 strain.

FIG. 10 shows the pH influence on α-isomaltosylglucosaccharide-forming enzyme from a microorganism of Bacillus globisporus C11 strain.

FIG. 11 shows the thermal stability of α-isomaltosylglucosaccharide-forming enzyme from a microorganism of Bacillus globisporus C11 strain.

FIG. 12 shows the pH stability of α-isomaltosylglucosaccharide-forming enzyme from a microorganism of Bacillus globisporus C11 strain.

FIG. 13 shows the thermal influence on the enzymatic activity of α-isomaltosyl-transferring enzyme from a microorganism of Bacillus globisporus C11 strain.

FIG. 14 shows the pH influence on the enzymatic activity of α-isomaltosyl-transferring enzyme from a microorganism of Bacillus globisporus C11 strain.

FIG. 15 shows the thermal stability of α-isomaltosyl-transferring enzyme from a microorganism of Bacillus globisporus C11 strain.

FIG. 16 shows the pH stability of α-isomaltosyl-transferring enzyme from a microorganism of Bacillus globisporus C11 strain.

FIG. 17 is a nuclear resonance spectrum (¹H-NMR) of α-isomaltosylmaltotriose, obtained by the enzymatic reaction using α-isomaltosylglucosaccharide-forming enzyme.

FIG. 18 is a nuclear resonance spectrum (¹H-NMR) of α-isomaltosylmaltotetraose, obtained by the enzymatic reaction using α-isomaltosylglucosaccharide-forming enzyme.

FIG. 19 is a nuclear resonance spectrum (¹³C-NMR) of α-isomaltosylmaltotriose, obtained by the enzymatic reaction using α-isomaltosylglucosaccharide-forming enzyme.

FIG. 20 is a nuclear resonance spectrum (¹³C-NMR) of α-isomaltosylmaltotetraose, obtained by the enzymatic reaction using α-isomaltosylglucosaccharide-forming enzyme.

FIG. 21 is a nuclear resonance spectrum (¹H-NMR) of the product A.

FIG. 22 is a nuclear resonance spectrum (¹³C-NMR) of the product A.

### BEST MODE FOR CARRYING OUT THE INVENTION

The α-isomaltosylglucosaccharide-forming enzyme usable in the present invention means an enzyme, which forms from amylaceous substances α-isomaltosylglucosaccharides such as α-isomaltosylglucose (alias panose), α-isomaltosylmaltose, α-isomaltosylmaltotriose, and α-isomaltosylmaltotetraose, and includes, for example, an α-isomaltosylglucosaccharide-forming enzyme from Bacillus globisporus C9, FERM BP-7143 (hereinafter may be designated as “Strain C9”), and Bacillus globisporus C11, FERM BP-7144 (hereinafter may be designated as “Strain C11”), which have been deposited on Apr. 25, 2000, in International Patent Organism Depositary National Institute of Advanced Industrial Science and Technology Tsukuba Central 6, 1-1, Higashi 1-Chome Tsukuba-shi, Ibaraki-ken, 305-8566, Japan, and which are disclosed in Japanese Patent Application No. 234,937/00; and recombinant polypeptides having an α-isomaltosylglucosaccharide-forming enzyme activity, disclosed in Japanese Patent Application No. 5,441/01.

The α-isomaltosyl-transferring enzyme usable in the present invention means an enzyme which forms cyclotetrasaccharide from α-isomaltosylglucosaccharides such as panose and α-isomaltosylmaltose: Examples of such include an α-isomaltosyl-transferring enzyme from Bacillus globisporus C9, FERM BP-7143, and Bacillus globisporus C11, FERM BP-7144, disclosed in Japanese Patent Application No. 229,557/00; and recombinant polypeptides having an α-isomaltosyl-transferring enzyme activity, disclosed in Japanese Patent Application No. 350,142/00.

The isomaltose-releasing enzyme usable in the present invention means an enzyme, which has an activity of releasing isomaltose from α-isomaltosylglucosaccharides or cyclotetrasaccharide, for example, isomaltodextranase (EC 3.2.1.94) from microorganisms such as Arthrobacter globiformis T6, NRRL B-4425, reported in Journal of Biochemistry, Vol. 75, pp. 105-112 (1974); Arthrobacter globiformis, IAM 12103, provided from Institute of Applied Microbiology (IAM), The University of Tokyo, Tokyo, Japan; and Actinomadura R10, NRRL B-11411, reported in Carbohydrate Research, Vol. 89, pp. 289-299 (1981).

The saccharides, which have both a glucose polymerization degree of at least two and α-1,4 glucosidic linkage as a linkage at the non-reducing end, usable in the present invention include, for example, terrestrial starches such as corns, rices, and wheats; and subterranean starches such as potatoes, sweet potatoes, and tapioca, as well as partial hydrolyzates thereof, i.e., partial starch hydrolyzates thereof. The partial starch hydrolyzates can be usually prepared by suspending the above terrestrial or subterranean starches in water, usually, into 10%, preferably, 15-65%, more preferably, 20-50% starch suspensions, and then liquefying the suspensions by heating or using acid agents or enzyme preparations. The degree of liquefaction is preferably set to a relatively low level, usually, less than DE (dextrose equivalent) 15, preferably, less than DE 10, and more preferably, DE 0.1-9. In the case of liquefaction with acid agents, there employed is a method comprising a step of liquefying the above starches with acid agents such as hydrochloric acid, phosphoric acid, and oxalic acid, and usually neutralizing the liquefied suspensions to the desired pHs with alkaline agents such as calcium carbonate, calcium oxide, and sodium carbonate. While in the case of liquefaction with enzyme preparations, α-amylases, particularly, thermostable liquefying α-amylases are preferably used in the present invention. Isomaltose can be obtained in a relatively high yield by allowing α-isomaltosylglucosaccharide-forming enzyme, in the presence or the absence of α-isomaltosyl-transferring enzyme, to act on saccharides, which have both a glucose polymerization degree of at least two and α-1,4 glucosidic linkage as a linkage at the non-reducing end, to form α-isomaltosylglucosaccharides, which have a glucose polymerization degree of at least three, α-1,6 glucosidic linkage as a linkage at the non-reducing end, and α-1,4 glucosidic linkage as a linkage other than the non-reducing end, and/or to form cyclo{→6)-α-D-glucopyranosyl-(1→3)-α-D-glucopyranosyl-(1→6)-α-D-glucopyranosyl-(1→3)-α-D-glucopyranosyl-(1→}; allowing isomaltosyl-releasing enzymes to act on the products to form isomaltose; and collecting the formed isomaltose. When allowed to act on substrates, α-isomaltosylglucosaccharide-forming enzyme can be used in combination with one or more another enzymes of α-isomaltosyl-transferring enzyme, cyclomaltodextrin glucanotransferase (hereinafter abbreviated as “CGTase”), α-glucosidase, glucoamylase, and starch debranching enzymes such as isoamylase and pullulanase to more increase the yield of isomaltose. Particularly, the yield of isomaltose from cyclotetrasaccharide can be increased up to a maximum yield of 100% in such a manner of allowing isomaltose-releasing enzyme to act on cyclotetrasaccharide obtained by allowing α-isomaltosylglucosaccharide-forming enzyme, in the presence of α-isomaltosyl-transferring enzyme, to act on saccharides having both a glucose polymerization degree of at least two and α-1,4 glucosidic linkage as a linkage at the non-reducing end. The order of a plurality of enzymes employed in the present invention can be decided depending on the yield of isomaltose, reaction times, reaction conditions, etc. These enzymes can be allowed to act on substrates at the same time or different timings after divided into several aliquots with the desired amount. Any pHs at which the enzymes used in the present invention are allowed to act on their substrates can be employed as long as the enzymes exert their enzymatic activities at the pHs, usually, those which are selected from pH 4-10, preferably, pH 5-8. The temperatures of allowing enzymes are usually selected from 10-80° C., preferably, 30-70° C. The amount of enzymes used can be appropriately altered in view of the reaction condition and time for each enzyme: Usually, the amounts of α-isomaltosyl-transferring enzyme and α-isomaltosylglucosaccharide-forming enzyme used are respectively selected from 0.01-100 units, the amounts of isomaltose-releasing enzyme and starch debranching enzyme used are selected from 1-10,000 units, and the amounts of CGTase, α-glucosidase, glucoamylase, and isoamylase used are selected from 0.05-7,000 units. Although the reaction time of enzymes used is varied depending on their amounts used, it is appropriately selected in view of the yield of isomaltose. Usually, the reaction time is set to 1-200 hours, preferably, 5-150 hours, and more preferably, 10-100 hours to complete the overall enzymatic reactions. The pHs and temperatures in the enzymatic reaction for each enzyme can be appropriately altered before termination of the enzymatic reactions of the present invention.

The content of isomaltose in the enzymatic reaction mixtures thus obtained is usually, on a dry solid basis, at least 30%, preferably, at least 40%, more preferably, at least 50%, and still more preferably, up to a maximum level of 99% or higher. Particularly, when α-isomaltosylglucosaccharide-forming enzyme, α-isomaltosyl-transferring enzyme, and isomaltose-releasing enzyme are simultaneously or in this order added to and allowed to act on saccharides having both a glucose polymerization degree of at least two and α-1,4 glucosidic linkage as a linkage at the non-reducing end, enzymatic reaction mixtures with an isomaltose content of at least 50%, d.s.b., can be easily obtained. In general, the reaction mixtures can be subjected to conventional methods such as filtration and centrifugation to remove impurities; decolored with an activated charcoal, desalted and purified, for example, by ion-exchange resins in a H— or OH-form; and concentrated into syrupy products; and optionally dried into powdery products. If necessary, the resulting products can be further purified into high isomaltose content products by appropriately using alone or in combination with two or more methods of column chromatographies such as ion-exchange column chromatography, column chromatography using an activated charcoal, and silica gel column chromatography; separation using organic solvents such as alcohols and acetone; and membrane separation. In particular, as an industrial-scale production method for high isomaltose content product, ion-exchange column chromatography is preferably employed. For example, high isomaltose content products can be produced on an industrial scale at a relatively high yield and amount and at a lesser cost by ion-exchange column chromatography using one or more styrene-divinylbenzene cross-linked copolymeric resins with sulfonyl group and strong-acid cation exchange resins in the form of alkaline metals such as Na⁺ and K⁺, and of alkaline earth metals such as Ca²⁺ and Mg²⁺, as disclosed in Japanese Patent Kokai Nos. 23,799/83 and 72,598/83. Examples of commercialized products of the above strong-acid cation exchange resins include “DOWEX 50WX2”, “DOWEX 50WX4”, and “DOWEX 50WX8”, produced by Dow Chemical Co., Midland, Mich., USA; “AMBERLITE CG-120”, produced by Rohm & Hass Company, Philadelphia, Pa., USA; “XT-1022E” produced by Tokyo Organic Chemical Industries, Ltd., Tokyo, Japan; and “DIAION SKLB”, “DIAION SK102”, and “DIAION SK104”, produced by Mitsubishi Chemical Industries, Tokyo, Japan. In practicing the above ion-exchange column chromatography, any one of fixed-bed, moving bed, and semi-moving bed methods can be appropriately used. With these methods the purity of isomaltose can be increased, usually, to at least 60%, preferably, at least 80%, and more preferably, at least 99%, d.s.b., as the highest possible purity. Products of isomaltose except for the highest possible isomaltose, i.e., high isomaltose content products usually contain isomaltose and 1-60%, d.s.b., of one or more saccharides from glucose, maltose, maltotriose, maltotetraose, other starch hydrolyzates, α-isomaltosylglucosaccharides, and α-glucosyl-(1→6)-α-glucosyl-(1→3)-α-glucosyl-(1→6)-α-glucose (may be abbreviated as “open-ring tetrasaccharide”, hereinafter).

The isomaltose and high isomaltose content products thus obtained can be suitably used as sweeteners which substantially do not induce dental caries because of their action of inhibiting the formation of dextran as a cause of dental caries, as well as satisfactory quality and good tastable sweetness. The isomaltose and high isomaltose content products of the present invention have also satisfactory storage stability. Particularly, products with a relatively high content of crystalline isomaltose can be advantageously used as sugar coatings for tablets in combination with conventional binders such as pullulan, hydroxyethyl starch, and polyvinylpyrrolidone. The isomaltose and high isomaltose content products of the present invention have useful properties of osmosis-controlling ability, filler-imparting ability, gloss-imparting ability, humectancy, viscosity, crystallization-preventing ability for saccharides, insubstantial fermentability, retrogradation-preventing ability for gelatinized starches, etc. Thus, the isomaltose and high isomaltose content products can be arbitrary used as a sweetener, taste-improving agent, flavor-improving agent, quality-improving agent, stabilizer, excipient, filler, etc., in a variety of compositions such as food products, feeds, pet foods, cosmetics, pharmaceuticals, tobaccos, and cigarettes.

The isomaltose and high isomaltose content products of the present invention can be used as seasonings to sweeten food products, and if necessary, they can be arbitrarily used in combination with one or more other sweeteners such as powdered syrup, glucose, fructose, lactosucrose, maltose, sucrose, isomerized sugar, honey, maple sugar, isomaltooligosaccharides, galactooligosaccharides, fructooligosaccharides, sorbitol, maltitol, lactitol, dihydrochalcone, stevioside, α-glycosyl stevioside, sweetener of Momordica grosvenori, glycyrrhizin, L-aspartyl L-phenylalanine methyl ester, sucralose, acesulfame K, saccharin, glycine, and alanine; and fillers such as dextrins, starches, and lactose.

Particularly, the isomaltose and high isomaltose content products of the present invention can be arbitrarily used intact or after mixing with appropriate fillers, excipients, binders, sweeteners, etc., and then formed into products with different shapes such as granules, spheres, plates, cubes, tablets, films, and sheets.

Since the isomaltose and high isomaltose content products of the present invention well harmonize with other tastable materials having sour-, acid-, salty-, astringent-, delicious-, or bitter-tastes, and have a satisfactorily high acid- and heat-tolerance, they can be favorably used in food products to sweeten and/or improve the taste or the quality of food products; amino acids, peptides, soy sauces, powdered soy sauces, miso, “funmatsu-miso” (a powdered miso), “moromi” (a refined sake), “hishio” (a refined soy sauce), “furikake” (a seasoned fish meal), mayonnaises, dressings, vinegars, “sanbai-zu” (a sauce of sugar, soy sauce and vinegar), “funmatsu-sushi-su” (powdered vinegar for sushi), “chuka-no-moto” (an instant mix for Chinese dish), “tentsuyu” (a sauce for Japanese deep-fat fried food), “mentsuyu” (a sauce for Japanese vermicelli), sauce, catsups, “yakiniku-no-tare” (a sauce for Japanese grilled meat), curry roux, instant stew mixes, instant soup mixes, “dashi-no-moto” (an instant stock mix), nucleotide seasonings, mixed seasonings, “mirin” (a sweet sake), “shin-mirin” (a synthetic mirin), table sugars, and coffee sugars. Also, the isomaltose and high isomaltose content products of the present invention can be arbitrarily used in “wagashi” (Japanese cakes) such as “senbei” (a rice cracker), “arare” (a rice cake cube), “okoshi” (a millet-and-rice cake), “mochi” (a rice paste) and the like, “manju” (a bun with a bean-jam), “uiro” (a sweet rice jelly), “an” (a bean jam) and the like, “yokan” (a sweet jelly of beans), “mizu-yokan” (a soft adzuki-bean jelly), “kingyoku” (a kind of yokan), jellies, pao de Castella, and “amedama” (a Japanese toffee); Western confectioneries such as a bun, biscuit, cracker, cookie, pie, pudding, butter cream, custard cream, cream puff, waffle, sponge cake, doughnut, chocolate, chewing gum, caramel, nougat, and candy; frozen desserts such as an ice cream and sherbet; syrups such as “kajitsu-no-syrup-zuke” (a preserved fruit) and “korimitsu” (a sugar syrup for shaved ice); pastes such as a flour paste, peanut paste, fruit paste, and spread; processed fruits and vegetables such as a jam, marmalade, “syrup-zuke” (fruit pickles), and “toka” (conserves); pickles and pickled products such as a “fukujin-zuke” (red colored radish pickles), “bettara-zuke” (a kind of whole fresh radish pickles), “senmai-zuke” (a kind of sliced fresh radish pickles), and “rakkyo-zuke” (pickled shallots); premixes for pickles and pickled products such as a “takuan-zuke-no-moto” (a premix for pickled radish), and “hakusai-zuke-no-moto” (a premix for fresh white rape pickles); meat products such as a ham and sausage; products of fish meat such as a fish ham, fish sausage, “kamaboko” (a steamed fish paste), “chikuwa” (a kind of fish paste), and “tenpura” (a Japanese deep-fat fried fish paste); “chinmi” (relish) such as a “uni-no-shiokara” (salted guts of sea urchin), “ika-no-shiokara” (salted guts of squid), “su-konbu” (processed tangle), “saki-surume” (dried squid strips), “fugu-no-mirin-boshi” (a dried mirin-seasoned swellfish); “tsukudani” (foods boiled down in soy sauce) such as those of lavers, edible wild plants, dried squids, small fishes, and shellfishes; daily dishes such as a “nimame” (cooked beans), potato salad, and “konbu-maki” (a tangle roll); milk products such as yogurts and cheeses; canned and bottled products such as those of meat, fish meat, fruit, and vegetables; alcoholic beverages such as a sake, synthetic sake, liqueur, and foreign liquors and drinks; soft drinks such as a coffee, tea, cocoa, juice, carbonated beverage, sour milk beverage, and beverage containing lactic acid bacteria; instant food products such as an instant pudding mix, instant hot cake mix, “sokuseki-shiruko” (an instant mix of adzuki-bean soup with rice cake), and instant soup mix; and other foods and beverages such as a solid food for babies, food for therapy, health/tonic drink, peptide food, frozen food, and health food.

The isomaltose and high isomaltose content products of the present invention can be arbitrarily used to improve the taste preference of feeds and pet foods for animals and pets such as domestic animals, poultry, honey bees, silk worms, and fishes; and also they can be arbitrary used as a sweetener, taste-improving agent, flavoring substance, quality-improving agent, and stabilizer in products in a liquid or solid form such as a tobacco, cigarette, tooth paste, lipstick/rouge, lip cream, internal liquid medicine, tablet, troche, cod liver oil in the form of drop, cachou, oral refrigerant, and gargle.

Stable and high-quality health foods and pharmaceuticals in a liquid, paste or solid form can be obtained by incorporating, as a quality-improving agent and/or stabilizer, the isomaltose and high isomaltose content products of the present invention into health foods and pharmaceuticals which contain effective ingredients, active ingredients, or biologically active substances. Examples of such biologically active substances include lymphokines such as α-, β- and γ-interferons, tumor necrosis factor-α (TNF-α), tumor necrosis factor-β (TNF-β), macrophage migration inhibitory factor, colony-stimulating factor, transfer factor, and interleukins; hormones such as insulin, growth hormone, prolactin, erythropoietin, and follicle-stimulating hormone; biological preparations such as BCG vaccine, Japanese encephalitis vaccine, measles vaccine, live polio vaccine, smallpox vaccine, tetanus toxoid, Trimeresurus antitoxin, and human immunoglobulin; antibiotics such as penicillin, erythromycin, chloramphenicol, tetracycline, streptomycin, and kanamycin sulfate; vitamins such as thiamine, riboflavin, L-ascorbic acid, α-glycosyl ascorbic acid, cod liver oil, carotenoid, ergosterol, tocopherol, rutin, α-glycosyl rutin, naringin, α-glycosyl naringin, hesperidin, and α-glycosyl hesperidin; enzymes such as lipase, elastase, urokinase, protease, β-amylase, isoamylase, glucanase, and lactase; extracts such as a ginseng extract, bamboo leaf extract, Japanese apricot extract, pine leaf extract, snapping turtle extract, chlorella extract, aloe extract, and propolis extract; live microorganisms such as viruses, lactic acid bacteria, and yeasts; and royal jelly.

The methods for incorporating the isomaltose or the high isomaltose content products of the present invention into the aforesaid compositions are those which can complete the incorporation before completion of the processings of the compositions, and can be appropriately selected from the following conventional methods of mixing, kneading, dissolving, melting, soaking, penetrating, dispersing, applying, coating, spraying, injecting, crystallizing, and solidifying. The isomaltose or the high isomaltose content product can be preferably incorporated into the compositions in an amount, usually, of at least 0.1%, preferably, at least 1%, and more preferably, 2-99.99% (w/w).

The following experiments explain the present invention in more detail:

Experiment 1

### Preparation of α-isomaltosylqlucosaccharide-forming enzyme and α-isomaltosyl-transferring enzyme

A liquid culture medium consisting of 4.0% (w/v) of “PINE-DEX.#4”, a partial starch hydrolysate commercialized by Matsutani Chemical Ind., Tokyo, Japan, 1.8% (w/v) of “ASAHIMEAST”, a yeast extract commercialized by Asahi Breweries, Ltd., Tokyo, Japan, 0.1% (w/v) of dipotassium phosphate, 0.06% (w/v) of sodium phosphate dodecahydrate, 0.05% (w/v) magnesium sulfate heptahydrate, and water was placed in 500-ml Erlenmeyer flasks in a respective volume of 100 ml, sterilized by autoclaving at 121° C. for 20 min, cooled, and then seeded with Bacillus globisporus C9 strain, FERM BP-7143, followed by culturing under rotary-shaking conditions at 27° C. and 230 rpm for 48 hours for a seed culture.

About 20 L of a fresh preparation of the same liquid culture medium as used in the above seed culture were placed in a 30-L fermentor, sterilized by heating, and then cooled to 27° C. and inoculated with 1% (v/v) of the seed culture, followed by culturing at 27° C. and pH 6.0-8.0 for 48 hours under aeration-agitation conditions. After completion of the culture, the resulting culture, which had about 0.45 unit/ml of the α-isomaltosylglucosaccharide-forming enzyme, about 1.5 units/ml of α-isomaltosyl-transferring enzyme, and about 0.95 unit/ml of cyclotetrasaccharide-forming activity, was centrifuged at 10,000 rpm for 30 min to obtain about 18 L of a supernatant. When measured for enzymatic activity, the supernatant had about 0.45 unit/ml of the α-isomaltosylglucosaccharide-forming enzyme, i.e., a total enzymatic activity of about 8,110 units; about 1.5 units/ml of α-isomaltosyl-transferring enzyme, i.e., a total enzymatic activity of about 26,900 units. The supernatant thus obtained can be used as an enzyme preparation of α-isomaltosylglucosaccharide-forming enzyme and α-isomaltosyl-transferring enzyme.

The activities of these enzymes were assayed as follows: The α-isomaltosylglucosaccharide-forming enzyme of the present invention was assayed for enzymatic activity by dissolving maltotriose in 100 mM acetate buffer (pH 6.0) to give a concentration of 2% (w/v) for a substrate solution, adding a 0.5 ml of an enzyme solution to a 0. 5 ml of the substrate solution, enzymatically reacting the mixture solution at 35° C. for 60 min, suspending the reaction mixture by heating at 100° C. for 10 min, and quantifying maltose, among the isomaltosyl maltose and maltose formed in the reaction mixture, by high-performance liquid chromatography (abbreviated as “HPLC” hereinafter). HPLC was carried out using “YMC PACK ODS-AQ303 column” commercialized by YMC Co., Ltd., Tokyo, Japan, at a column temperature of 40° C. and a flow rate of 0.5 ml/min of water; and using “RI-8012”, a differential refractometer commercialized by Tosoh Corporation, Tokyo, Japan. One unit activity of the α-isomaltosylglucosaccharide-forming enzyme is defined as the enzyme amount that forms one micromole of maltose per minute under the above enzymatic reaction conditions.

The α-isomaltosyl-transferring enzyme was assayed for enzymatic activity by dissolving panose in 100 mM acetate buffer (pH 6.0) to give a concentration of 2% (w/v) for a substrate solution, adding a 0.5 ml of an enzyme solution to 0.5 ml of the substrate solution, enzymatically reacting the mixture solution at 35° C. for 30 min, suspending the reaction mixture by boiling for 10 min, and quantifying glucose, among the cyclotetrasaccharide and glucose formed in the reaction mixture, by the glucose oxidase method. One unit activity of the α-isomaltosyl-transferring enzyme is defined as the enzyme amount that forms one micromole of glucose per minute under the above enzymatic reaction conditions.

The cyclotetrasaccharide-forming activity is assayed by dissolving “PINE-DEX #100”, a partial starch hydrolysate commercialized by Matsutani Chemical Ind., Tokyo, Japan, in 50 mM acetate buffer (pH 6.0) to give a concentration of 2% (w/v) for a substrate solution, adding 0.5 ml of an enzyme solution to 0.5 ml of the substrate solution, enzymatically reacting the mixture solution at 35° C. for 60 min, suspending the reaction mixture by boiling for 10 min, and then further adding to the resulting mixture one milliliter of 50 mM acetate buffer (pH 5.0) with 70 units/ml of “TRANSGLUCOSIDASE L AMANO™”, an α-glucosidase commercialized by Amano Pharmaceutical Co., Ltd., Aichi, Japan, and 27 units/ml of glucoamylase, commercialized by Nagase Biochemicals, Ltd., Kyoto, Japan, and incubated at 50° C. for 60 min, inactivating the remaining enzymes by heating at 100° C. for 10 min, and quantifying cyclotetrasaccharide on HPLC similarly as above. One unit of cyclotetrasaccharide-forming activity is defined as the enzyme amount that forms one micromole of cyclotetrasaccharide per minute under the above enzymatic reaction conditions.

Experiment 2

### Isolation of α-isomaltosylglucosaccharide-forming enzyme and α-isomaltosyl-transferring enzyme

Experiment 2-1

### Isolation of α-isomaltosylglucosaccharide-forming enzyme

About 18 L of the supernatant in Experiment 1 was salted out with 80% saturated ammonium sulfate and allowed to stand at 4° C. for 24 hours, and the formed precipitates were collected by centrifugation at 10,000 rpm for 30 min, dissolved in 10 mM phosphate buffer (pH 7.5), and dialyzed against a fresh preparation of the same buffer to obtain about 400 ml of a crude enzyme solution with 8,110 units of α-isomaltosylglucosaccharide-forming enzyme, 24,700 units of α-isomaltosyl-transferring enzyme, and about 15,600 units of cyclotetrasaccharide-forming activity. The crude enzyme solution was subjected to ion-exchange chromatography using 1,000 ml of “SEPABEADS FP-DA13” gel, an ion-exchange resin commercialized by Mitsubishi Chemical Industries, Ltd., Tokyo, Japan. The α-isomaltosylglucosaccharide-forming enzyme and cyclotetrasaccharide were eluted as non-adsorbed fractions without adsorbing on the ion-exchange resin. The resulting enzyme solution was dialyzed against 10 mM phosphate buffer (pH 7.0) with 1 M ammonium sulfate, and the dialyzed solution was centrifuged to remove impurities, and subjected to affinity chromatography using 500 ml of “SEPHACRYL HR S-200”, a gel commercialized by Amersham Corp., Div. Amersham International, Arlington Heights, Ill., USA. Enzymatically active components adsorbed on the gel and, when sequentially eluted with a linear gradient decreasing from 1 M to 0 M of ammonium sulfate and a linear gradient increasing from 0 mM to 100 mM of maltotetraose, the α-isomaltosylglucosaccharide-forming enzyme and the α-isomaltosyl-transferring enzyme were separately eluted, i.e., the former was eluted with the linear gradient of maltotetraose at about 30 mM and the latter was eluted with the linear gradient of ammonium sulfate at about 0 M. Thus, fractions with α-isomaltosyl-transferring activity and those with the α-isomaltosylglucosaccharide-forming activity were separatory collected.

The above α-isomaltosylglucosaccharide-forming enzyme fraction were pooled and dialyzed against 10 mM phosphate buffer (pH 7.0) containing 1 M ammonium sulfate. The dialyzed solution was centrifuged to remove insoluble impurities, and the resulting supernatant was fed to hydrophobic chromatography using 350 ml of “BUTYL-TOYOPEARL 650 M”, a gel commercialized by Tosoh Corporation, Tokyo, Japan. The active enzyme was adsorbed on the gel and then eluted therefrom at about 0.3 M ammonium sulfate using a linear gradient decreasing from 1 M to 0 M of ammonium sulfate, followed by collecting fractions with the enzyme activity. The fractions were pooled and then dialyzed against 10 mM phosphate buffer (pH 7.0) containing 1 M ammonium sulfate. The resulting dialyzed solution was centrifuged to remove impurities and fed to affinity chromatography using “SEPHACRYL HR S-200” gel to purify the enzyme. The amount of enzyme activity, specific activity, and yield of the α-isomaltosylglucosaccharide-forming enzyme in each purification step are in Table 1.

TABLE 1

| | | Specific activity | |
|---|---|---|---|
| | Enzyme* activity | of enzyme* | Yield |
| Purification step | (unit) | (unit/mg protein) | (%) |
| Culture supernatant | 8,110 | 0.12 | 100 |
| Dialyzed solution after | 7,450 | 0.56 | 91.9 |
| salting out with ammonium | | | |
| sulfate | | | |
| Eluate from ion-exchange | 5,850 | 1.03 | 72.1 |
| column chromatography | | | |
| Eluate from affinity | 4,040 | 8.72 | 49.8 |
| column chromatography | | | |
| Eluate from hydrophobic | 3,070 | 10.6 | 37.8 |
| column chromatography | | | |
| Eluate from affinity | 1,870 | 13.6 | 23.1 |
| column chromatography | | | |

Note:
The symbol “*” means the α-isomaltosylglucosaccharide-forming enzyme.

The finally purified α-isomaltosylglucosaccharide-forming enzyme specimen was assayed for purity on gel electrophoresis using a 7.5% (w/v) polyacrylamide gel and detected on the gel as a single protein band, i.e., a high purity enzyme specimen.

Experiment 2-2

### Property of α-isomaltosylglucosaccharide-forming enzyme

A purified specimen of α-isomaltosylglucosaccharide-forming enzyme, obtained by the method in Experiment 2-1, was subjected to SDS-PAGE using a 7.5% (w/v) of polyacrylamide gel and then determined for molecular weight by comparing with the dynamics of standard molecular markers electrophoresed in parallel, commercialized by Bio-Rad Laboratories Inc., Brussels, Belgium, revealing that the enzyme had a molecular weight of about 140,000±20,000 daltons.

A fresh preparation of the above purified specimen was subjected to isoelectrophoresis using a gel containing 2% (w/v) ampholine commercialized by Amersham Corp., Div. Amersham International, Arlington Heights, Ill., USA, and then measured for pHs of protein bands and gels to determine the isoelectric point of the enzyme, revealing that the enzyme had an isoelectric point of about 5.2±0.5.

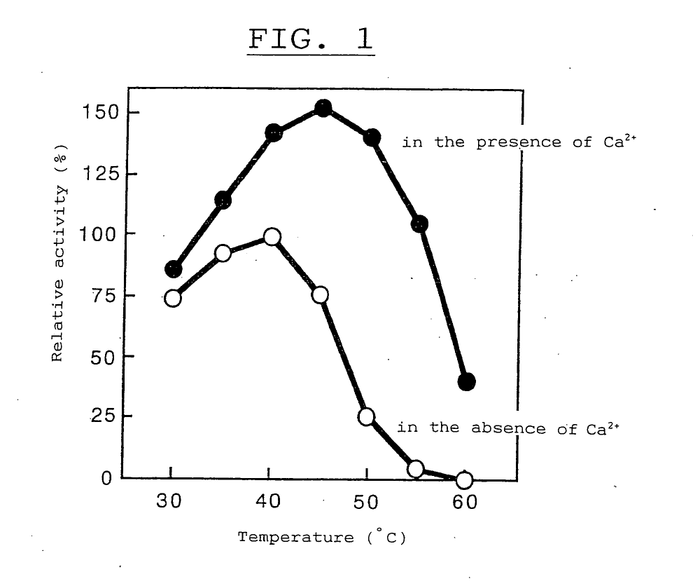

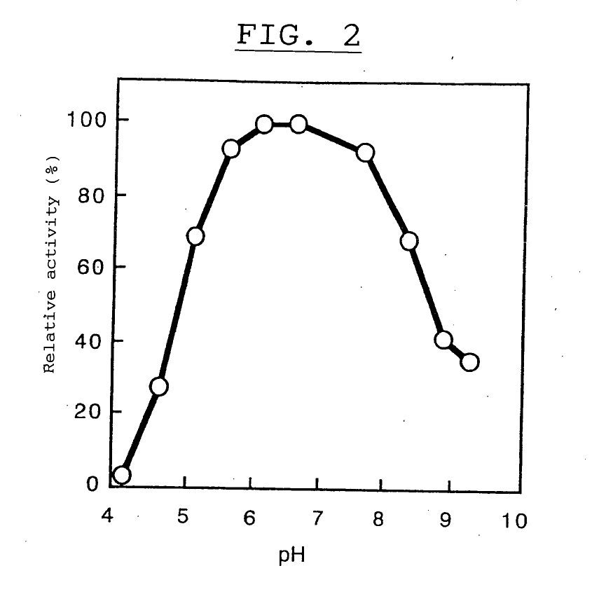

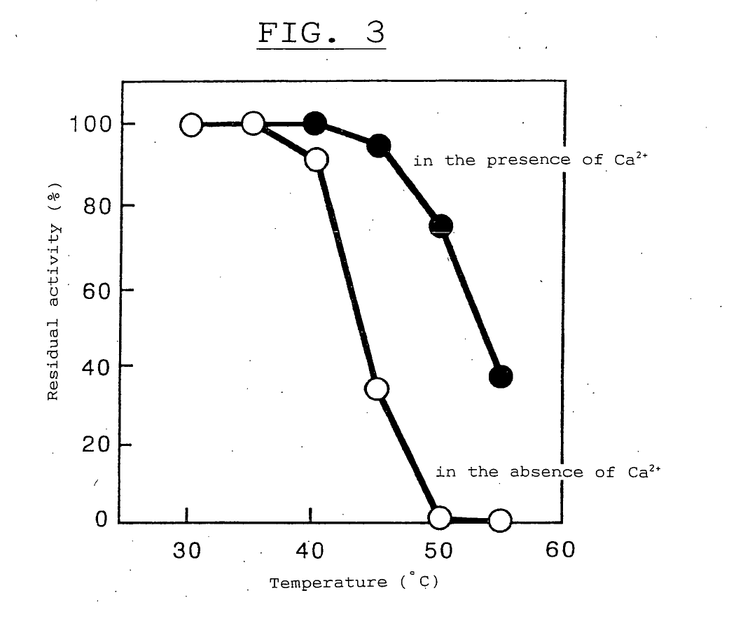

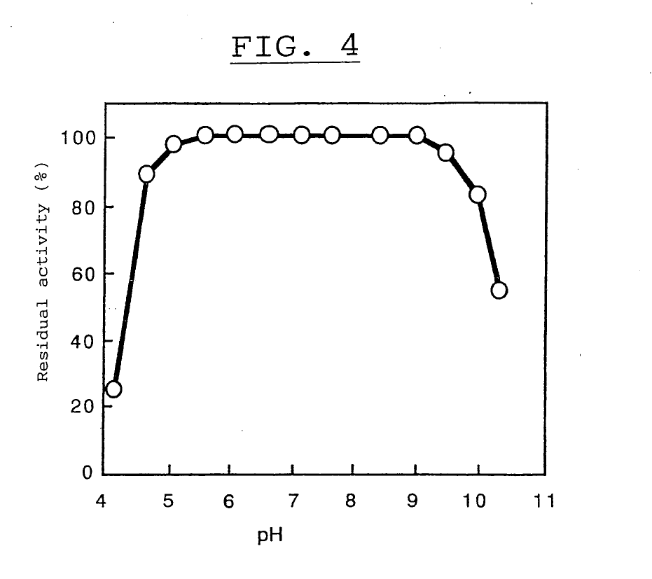

The influence of temperature and pH on the activity of α-isomaltosylglucosaccharide-forming enzyme was examined in accordance with the assay for the enzyme activity, where the influence of temperature was conducted in the presence or the absence of 1 mM Ca²⁺. These results are in FIG. 1 (influence of temperature) and FIG. 2 (influence of pH). The optimum temperature of the enzyme was about 40° C. (in the absence of Ca²⁺) and about 45° C. (in the presence of 1 mM Ca²⁺) when incubated at pH 6.0 for 60 min, and the optimum pH of the enzyme was about 6.0 to about 6.5 when incubated at 35° C. for 60 min. The thermal stability of the enzyme was determined by incubating the testing enzyme solutions in 20 mM acetate buffer (pH 6.0) at prescribed temperatures for 60 min in the presence or the absence of 1 mM Ca²⁺, cooling the resulting enzyme solutions with water, and assaying the remaining enzyme activity of each solution. The pH stability of the enzymes was determined by keeping the testing enzyme solutions in 50 mM buffers having prescribed pHs at 4° C. for 24 hours, adjusting the pH of each solution to 6.0, and assaying the remaining enzyme activity of each solution. These results are respectively in FIG. 3 (thermal stability) and FIG. 4 (pH stability). As a result, the enzyme had thermal stability of up to about 35° C. in the absence of Ca²⁺ and about 40° C. in the presence of 1 mM Ca²⁺, and pH stability of about 4.5 to about 9.0.

The influence of metal ions on the activity of α-isomaltosylglucosaccharide-forming enzyme was examined in the presence of 1 mM of each metal-ion according to the assay for the enzyme activity. The results are in Table 2.

TABLE 2

| | Relative activity | | Relative activity |
|---|---|---|---|
| Metal ion | (%) | Metal ion | (%) |
| None | 100 | Hg²⁺ | 4 |
| Zn²⁺ | 92 | Ba²⁺ | 65 |
| Mg²⁺ | 100 | Sr²⁺ | 80 |
| Ca²⁺ | 115 | Pb²⁺ | 103 |
| Co²⁺ | 100 | Fe²⁺ | 98 |
| Cu²⁺ | 15 | Fe³⁺ | 97 |
| Ni²⁺ | 98 | Mn²⁺ | 111 |
| Al³⁺ | 99 | EDTA | 20 |

As evident form the results in Table 2, the enzyme activity was greatly inhibited by Hg²⁺, Cu²⁺, and EDTA, and also inhibited by Ba²⁺ and Sr²⁺. It was also found that the enzyme was activated by Ca²⁺ and Mn²⁺.

Experiment 2-3

### Property of α-isomaltosyl-transferring enzyme

A fraction with α-isomaltosyl-transferring enzyme, obtained in Experiment 2-1, was dialyzed against 10 mM phosphate buffer (pH 7.0) containing 1 M ammonium sulfate. The resulting dialyzed solution was centrifuged to remove impurities and subjected to hydrophobic chromatography using 350 ml of “BUTYL-TOYOPEARL 650 M”, a gel commercialized by Tosoh Corporation, Tokyo, Japan. The enzyme adsorbed on the gel and then eluted with a linear gradient decreasing from 1 M to 0 M ammonium sulfate, resulting in an elution of the enzyme from the gel at a concentration of about 0.3 M ammonium sulfate and collecting fractions with the enzyme activity. Thereafter, the fractions were pooled and dialyzed against 10 mM phosphate buffer (pH 7.0) containing 1 M ammonium sulfate, and the dialyzed solution was centrifuged to remove impurities and purified on affinity chromatography using “SEPHACRYL HR S-200” gel. The amount of enzyme activity, specific activity, and yield of the α-isomaltosyl-transferring enzyme in each purification step are in Table 3.

TABLE 3

| | | Specific activity | |
|---|---|---|---|
| | Enzyme* activity | of enzyme* | Yield |
| Purification step | (unit) | (unit/mg protein) | (%) |
| Culture supernatant | 26,900 | 0.41 | 100 |
| Dialyzed solution after | 24,700 | 1.85 | 91.8 |
| salting out with ammonium | | | |
| sulfate | | | |
| Eluate from ion-exchange | 19,400 | 3.41 | 72.1 |
| column chromatography | | | |
| Eluate from affinity | 13,400 | 18.6 | 49.8 |
| column chromatography | | | |
| Eluate from hydrophobic | 10,000 | 21.3 | 37.2 |
| column chromatography | | | |
| Eluate from affinity | 6,460 | 26.9 | 24.0 |
| column chromatography | | | |

Note:
The symbol “*” means the α-isomaltosyl-transferring enzyme.

Experiment 2-4

### Property of α-isomaltosyl-transferring enzyme

The purified specimen of α-isomaltosyl-transferring enzyme in Experiment 2-3 was subjected to SDS-PAGE using a 7.5% (w/v) of polyacrylamide gel and then determined for molecular weight by comparing with the dynamics of standard molecular markers electrophoresed in parallel, commercialized by Bio-Rad Laboratories Inc., Brussels, Belgium, revealing that the enzyme had a molecular weight of about 112,000±20,000 daltons.

A fresh preparation of the above purified specimen was subjected to isoelectrophoresis using a gel containing 2% (w/v) ampholine commercialized by Amersham Corp., Div., Amersham International, Arlington Heights, Ill., USA, and then measured for pHs of protein bands and gel to determine the isoelectric point of the enzyme, revealing that the enzyme had an isoelectric point of about 5.5±0.5.

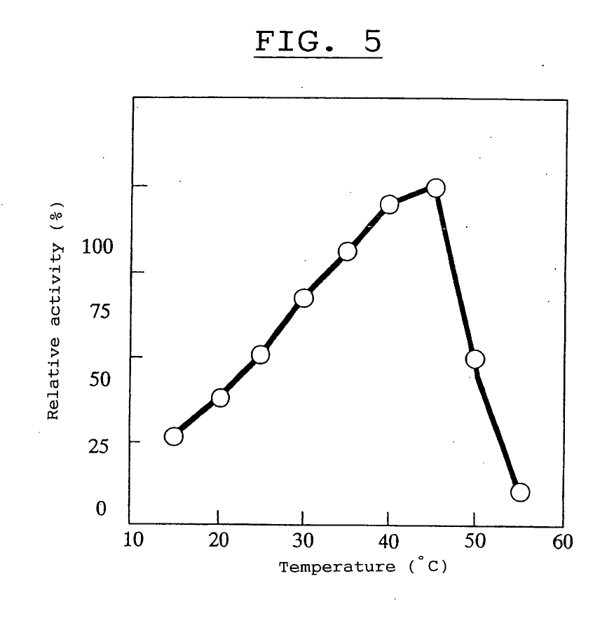

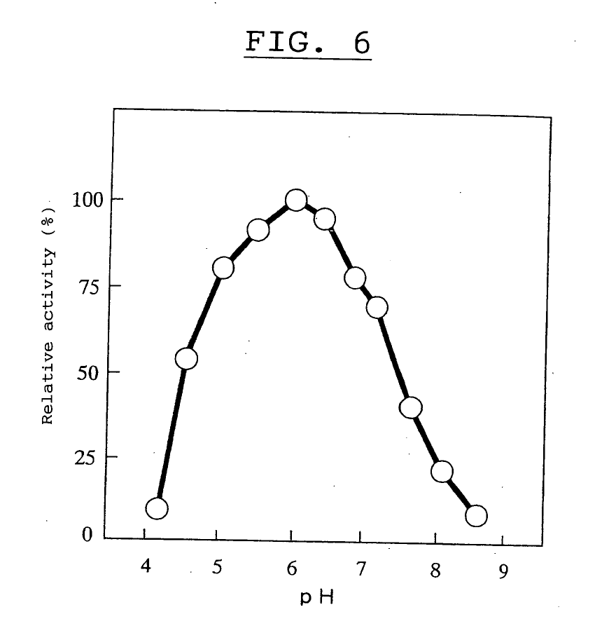

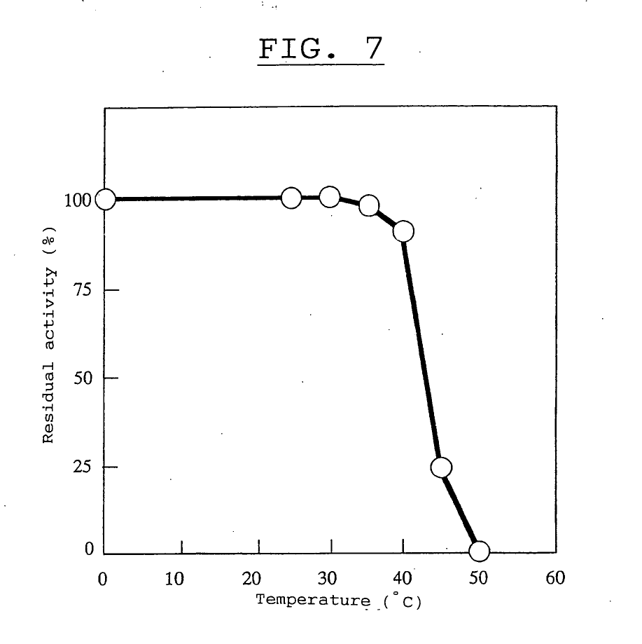

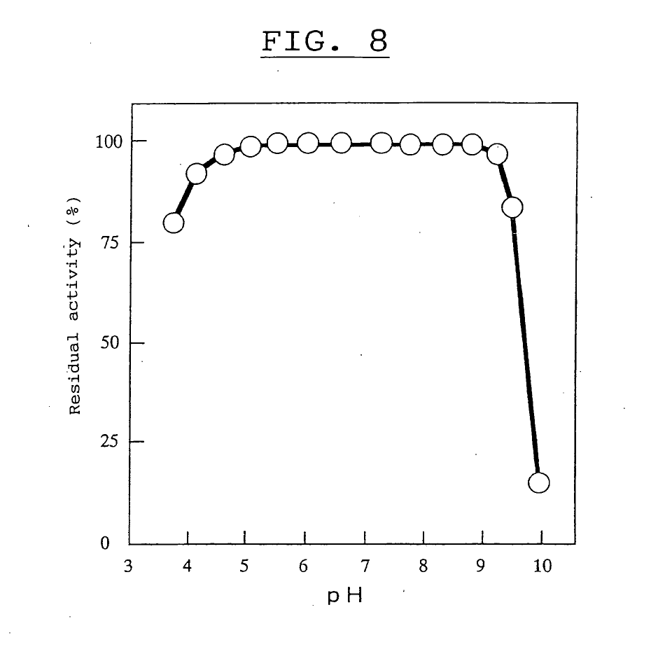

The influence of temperature and pH on the activity of α-isomaltosyl-transferring enzyme was examined in accordance with the assay for the enzyme activity. These results are in FIG. 5 (influence of temperature) and FIG. 6 (influence of pH). The optimum temperature of the enzyme was about 45° C. when incubated at pH 6.0 for 30 min, and the optimum pH of the enzyme was about 6.0 when incubated at 35° C. for 30 min. The thermal stability of the enzyme was determined by incubating the testing enzyme solutions in 20 mM acetate buffer (pH 6.0) at prescribed temperatures for 60 min, cooling the resulting enzyme solutions with water, and assaying the remaining enzyme activity of each solution. The pH stability of the enzyme was determined by keeping the testing enzyme solutions in 50 mM buffers having prescribed pHs at 4° C. for 24 hours, adjusting the pH of each solution to 6.0, and assaying the remaining enzyme activity of each solution. These results are respectively in FIG. 7 (thermal stability) and FIG. 8 (pH stability). As a result, the enzyme had thermal stability of up to about 40° C. and pH stability of about 4.0 to about 9.0.

The influence of metal ions on the activity of α-isomaltosyl-transferring enzyme was examined in the presence of 1 mM of each metal-ion according to the assay for the enzyme activity. The results are in Table 4.

TABLE 4

| | Relative activity | | Relative activity |
|---|---|---|---|
| Metal ion | (%) | Metal ion | (%) |
| None | 100 | Hg²⁺ | 1 |
| Zn²⁺ | 88 | Ba²⁺ | 102 |
| Mg²⁺ | 98 | Sr²⁺ | 101 |
| Ca²⁺ | 101 | Pb²⁺ | 89 |
| Co²⁺ | 103 | Fe²⁺ | 96 |
| Cu²⁺ | 57 | Fe³⁺ | 105 |
| Ni²⁺ | 102 | Mn²⁺ | 106 |
| Al³⁺ | 103 | EDTA | 104 |

As evident form the results in Table 4, the enzyme activity was greatly inhibited by Hg²+and also inhibited by Cu²⁺. It was also found that the enzyme was not activated by Ca²⁺ and not inhibited by EDTA.

Both the α-isomaltosylglucosaccharide-forming enzyme and the α-isomaltosyl-transferring enzyme from Bacillus globisporus C9 strain, FERM BP-7143, can be suitably used in the present invention.

Experiment 3

### Production of α-isomaltosylglucosaccharide-forming enzyme and α-isomaltosyl-transferring enzyme

A liquid nutrient culture medium, consisting of 4.0% (w/v) of “PINE-DEX #4”, a partial starch hydrolysate, 1.8% (w/v) of “ASAHIMEAST”, a yeast extract, 0.1% (w/v) of dipotassium phosphate, 0.06% (w/v) of sodium phosphate dodecahydrate, 0.05% (w/v) magnesium sulfate heptahydrate, and water was placed in 500-ml Erlenmeyer flasks in a respective volume of 100 ml each, autoclaved at 121° C. for 20 minutes to effect sterilization, cooled, inoculated with a stock culture of Bacillus globisporus C11, FERM BP-7144, and incubated at 27° C. for 48 hours under rotary shaking conditions of 230 rpm. The resulting cultures were pooled and used as a seed culture.

About 20 L of a fresh preparation of the same nutrient culture medium as used in the above culture were placed in a 30-L fermentor, sterilized by heating, cooled to 27° C., inoculated with 1% (v/v) of the seed culture, and incubated for about 48 hours while stirring under aeration agitation conditions at 27° C. and pH 6.0-8.0. The resultant culture, having about 0.55 unit/ml of α-isomaltosylglucosaccharide-forming enzyme activity, about 1.8 units/ml of α-isomaltosyl-transferring enzyme activity, and about 1.1 units/ml of cyclotetrasaccharide-forming enzyme activity, was centrifuged at 10,000 rpm for 30 min to obtain about 18 L of a supernatant. Measurement of the supernatant revealed that it had about 0.51 unit/ml of α-isomaltosylglucosaccharide-forming enzyme activity, i.e., a total enzyme activity of about 9,180 units; and about 1.7 units/ml of α-isomaltosyl-transferring enzyme activity, i.e., a total enzyme activity of about 30,400 units.

An 18 L of the above supernatant was salted out with an 80% saturated ammonium sulfate solution and allowed to stand at 4° C. for 24 hours. Then the salted out precipitates were collected by centrifugation at 10,000 for 30 min, dissolved in 10 mM phosphate buffer (pH 7.5), dialyzed against a fresh preparation of the same buffer to obtain about 416 ml of a crude enzyme solution. The crude enzyme solution was revealed to have 8,440 units of the α-isomaltosylglucosaccharide-forming enzyme, about 28,000 units of α-isomaltosyl-transferring enzyme, and about 17,700 units of cyclotetrasaccharide-forming enzyme. When subjected to ion-exchange chromatography using “SEPABEADS FP-DA13” gel, disclosed in Experiment 2-1, the above three types of enzymes were eluted as non-adsorbed fractions without adsorbing on the gel. The non-adsorbed fractions with those enzymes were pooled and dialyzed against 10 mM phosphate buffer (pH 7.0) containing 1 M ammonium sulfate, and the dialyzed solution was centrifuged to remove impurities. The resulting supernatant was fed to affinity chromatography using 500 ml of “SEPHACRYL HR S-200” gel to purify the enzyme. Active enzymes was adsorbed on the gel and was sequentially eluted with a linear gradient decreasing from 1 M to 0 M of ammonium sulfate and a linear gradient increasing from 0 mM to 100 mM of maltotetraose, followed by separate elutions of α-isomaltosyl-transferring enzyme and α-isomaltosylglucosaccharide-forming enzyme, where the former enzyme was eluted with the linear gradient of ammonium sulfate at a concentration of about 0.3 M and the latter enzyme was eluted with a linear gradient of maltotetraose at a concentration of about 30 mM. Therefore, fractions with the α-isomaltosylglucosaccharide-forming enzyme and those with α-isomaltosyl-transferring enzyme were separately collected and recovered.

Experiment 4

### Isolation of α-isomaltosylglucosaccharide-forming enzyme and α-isomaltosyl-transferring enzyme

The pooled fraction of α-isomaltosylglucosaccharide-forming enzyme in Experiment 3 was dialyzed against 10 mM phosphate buffer (pH 7.0) containing 1 M ammonium sulfate. The dialyzed solution was centrifuged to remove insoluble impurities, and the resulting supernatant was fed to hydrophobic chromatography using 350 ml of “BUTYL-TOYOPEARL 650 M”, a gel commercialized by Tosoh Corporation, Tokyo, Japan. The enzyme adsorbed on the gel was eluted therefrom at about 0.3 M ammonium sulfate using a linear gradient decreasing from 1 M to 0 M of ammonium sulfate, followed by collecting fractions with the enzyme activity. The fractions were pooled and dialyzed against 10 mM phosphate buffer (pH 7.0) containing 1 M ammonium sulfate. The resulting dialyzed solution was centrifuged to remove impurities and fed to affinity chromatography using “SEPHACRYL HR S-200” gel to purify the enzyme. The amount of enzyme activity, specific activity, and yield of the α-isomaltosylglucosaccharide-forming enzyme in each purification step are in Table 5.

TABLE 5

| | | Specific activity | |
|---|---|---|---|
| | Enzyme* activity | of enzyme* | Yield |
| Purification step | (unit) | (unit/mg protein) | (%) |
| Culture supernatant | 9,180 | 0.14 | 100 |
| Dialyzed solution after | 8,440 | 0.60 | 91.9 |
| salting out with ammonium | | | |
| sulfate | | | |
| Eluate from ion-exchange | 6,620 | 1.08 | 72.1 |
| column chromatography | | | |
| Eluate from affinity | 4,130 | 8.83 | 45.0 |
| column chromatography | | | |
| Eluate from hydrophobic | 3,310 | 11.0 | 36.1 |
| column chromatography | | | |
| Eluate from affinity | 2,000 | 13.4 | 21.8 |
| column chromatography | | | |

Note:
The symbol “*” means the α-isomaltosylglucosaccharide-forming enzyme.

The finally purified α-isomaltosylglucosaccharide-forming enzyme specimen was assayed for purity on gel electrophoresis using a 7.5% (w/v) polyacrylamide gel and detected on the gel as a single protein band, meaning a high purity enzyme specimen.

Experiment 4-2

### Property of α-isomaltosylglucosaccharide-forming enzyme

The purified specimen of α-isomaltosylglucosaccharide-forming enzyme in Experiment 4-1 was subjected to SDS-PAGE using a 7.5% (w/v) of polyacrylamide gel and then determined for molecular weight by comparing with the dynamics of standard molecular markers electrophoresed in parallel, commercialized by Bio-Rad Laboratories Inc., Brussels, Belgium, revealing that the enzyme had a molecular weight of about 137,000±20,000 daltons.

A fresh preparation of the above purified specimen was subjected to isoelectrophoresis using a gel containing 2% (w/v) ampholine commercialized by Amersham Corp., Div., Amersham International, Arlington Heights, Ill., USA, and then measured for pHs of protein bands and gel to determine the isoelectric point of the enzyme, revealing that the enzyme had an isoelectric point of about 5.2±0.5.

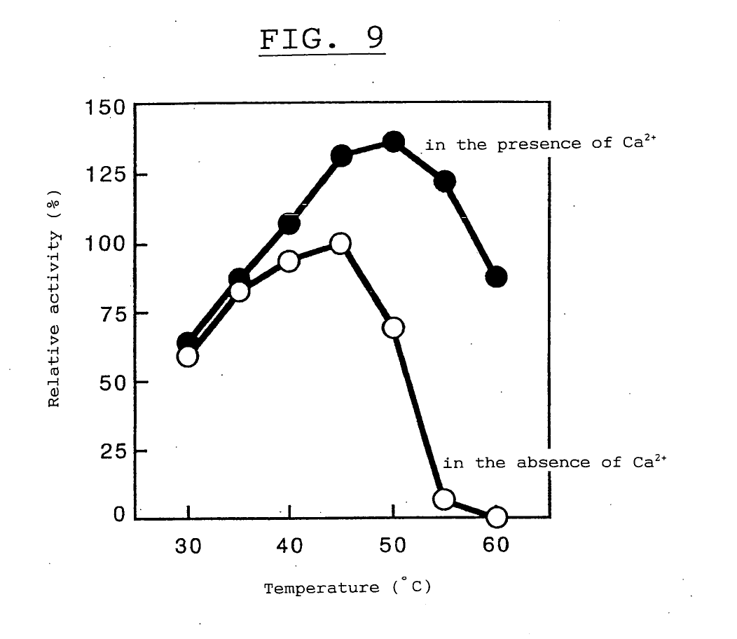

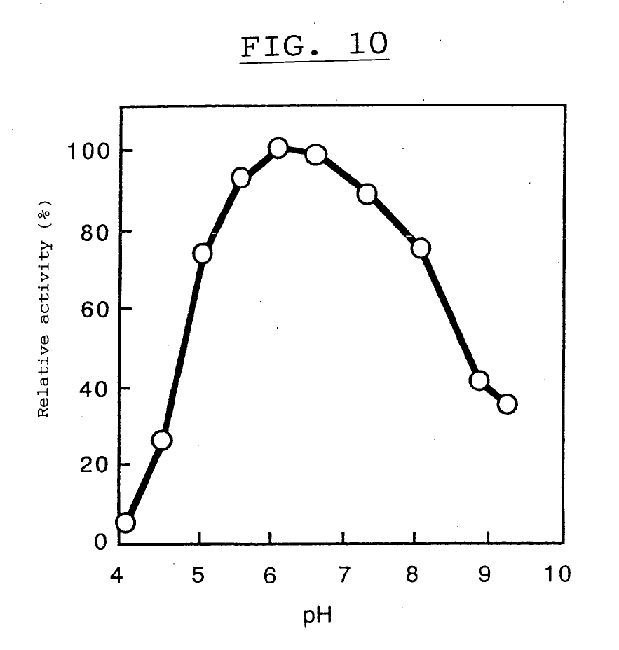

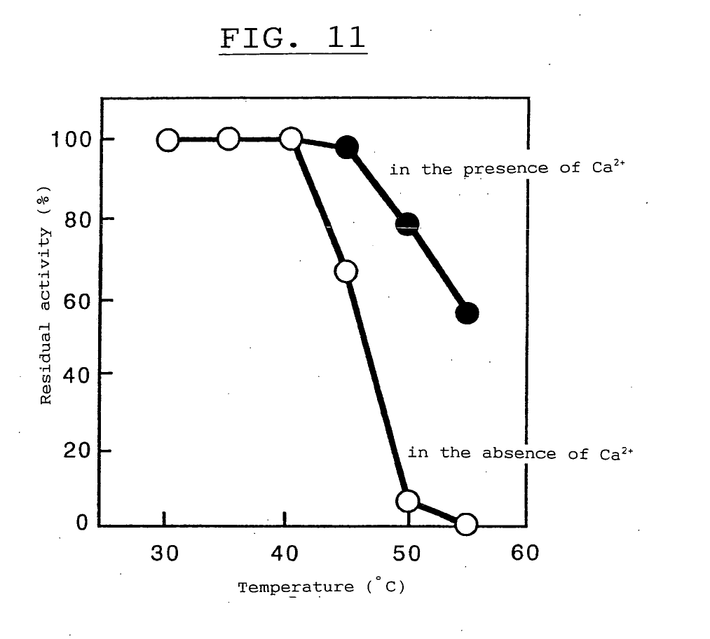

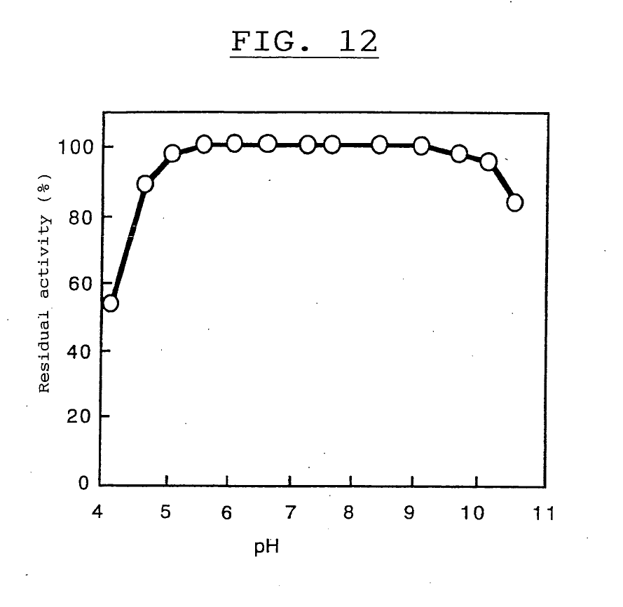

The influence of temperature and pH on the activity of α-isomaltosylglucosaccharide-forming enzyme was examined in accordance with the assay for the enzyme activity, where the influence of temperature was conducted in the presence or the absence of 1 mM Ca²⁺. These results are in FIG. 9 (influence of temperature) and FIG. 10 (influence of pH). The optimum temperature of the enzyme was about 45° C. in the absence of Ca²⁺ and about 50° C. in the presence of 1 mM Ca²⁺ when incubated at pH 6.0 for 60 min. The optimum pH of the enzyme was about 6.0 when incubated at 35° C. for 60 min. The thermal stability of the enzyme was determined by incubating the testing enzyme solutions in 20 mM acetate buffer (pH 6.0) in the presence or the absence of 1 mM Ca²⁺ at prescribed temperatures for 60 min, cooling the resulting enzyme solutions with water, and assaying the remaining enzyme activity of each solution. The pH stability of the enzyme was determined by keeping the testing enzyme solutions in 50 mM buffers having prescribed pHs at 4° C. for 24 hours, adjusting the pH of each solution to 6.0, and assaying the remaining enzyme activity of each solution. These results are respectively in FIG. 11 (thermal stability) and FIG. 12 (pH stability). As a result, the enzyme had thermal stability of up to about 40° C. in the absence of Ca²⁺ and up to about 45° C. in the presence of 1 mM Ca²⁺. The pH stability of enzyme was about 5.0 to about 10.0.

The influence of metal ions on the activity of α-isomaltosyl-transferring enzyme was examined in the presence of 1 mM of each metal-ion according to the assay for the enzyme activity. The results are in Table 6.

TABLE 6

| | Relative activity | | Relative activity |
|---|---|---|---|
| Metal ion | (%) | Metal ion | (%) |
| None | 100 | Hg²⁺ | 4 |
| Zn²⁺ | 91 | Ba²⁺ | 65 |
| Mg²⁺ | 98 | Sr²⁺ | 83 |
| Ca²⁺ | 109 | Pb²⁺ | 101 |
| Co²⁺ | 96 | Fe²⁺ | 100 |
| Cu²⁺ | 23 | Fe³⁺ | 102 |
| Ni²⁺ | 93 | Mn²⁺ | 142 |
| Al³⁺ | 100 | EDTA | 24 |

As evident form the results in Table 6, the enzyme activity was greatly inhibited by Hg²⁺, Cu²⁺, and EDTA and also inhibited by Ba²⁺ and Sr²⁺. It was also found that the enzyme was activated by Ca²⁺ and Mn²⁺.

Experiment 4-3

### Amino acid sequence of α-isomaltosylqlucosaccharide-forming enzyme

The specification does not describe in detail the method for analyzing the amino acid sequence of α-isomaltosylglucosaccharide-forming enzyme because it is disclosed in detail in Japanese Patent Application No. 5,441/01. Similarly as the polypeptide disclosed in the application, the α-isomaltosylglucosaccharide-forming enzyme in Experiment 4-1 has an amino acid sequence of the residues 36-1284 of SEQ ID NO:1 shown in parallel with nucleosides.

Experiment 4-4

### Isolation of α-isomaltosyl-transferring enzyme

The faction of α-isomaltosyl-transferring enzyme in Experiment 3 was dialyzed against 10 mM phosphate buffer (pH 7.0) containing 1 M ammonium sulfate. The dialyzed solution was centrifuged to remove insoluble impurities, and the resulting supernatant was fed to hydrophobic chromatography using 350 ml of “BUTYL-TOYOPEARL 650 M”, a gel commercialized by Tosoh Corporation, Tokyo, Japan. The enzyme adsorbed on the gel and then eluted therefrom at about 0.3 M ammonium sulfate using a linear gradient decreasing from 1 M to 0 M of ammonium sulfate, followed by collecting fractions with the enzyme activity. The fractions were pooled and dialyzed against 10 mM phosphate buffer (pH 7.0) containing 1 M ammonium sulfate. The resulting dialyzed solution was centrifuged to remove impurities and fed to affinity chromatography using “SEPHACRYL HR S-200” gel to purify the enzyme. The amount of enzyme activity, specific activity, and yield of the α-isomaltosyl-transferring enzyme in each purification step are in Table 7.

TABLE 7

| | | Specific activity | |
|---|---|---|---|
| | Enzyme* activity | of enzyme* | Yield |
| Purification step | (unit) | (unit/mg protein) | (%) |
| Culture supernatant | 30,400 | 0.45 | 100 |
| Dialyzed solution after | 28,000 | 1.98 | 92.1 |
| salting out with ammonium | | | |
| sulfate | | | |
| Eluate from ion-exchange | 21,800 | 3.56 | 71.7 |
| column chromatography | | | |
| Eluate from affinity | 13,700 | 21.9 | 45.1 |
| column chromatography | | | |
| Eluate from hydrophobic | 10,300 | 23.4 | 33.9 |
| column chromatography | | | |
| Eluate from affinity | 5,510 | 29.6 | 18.1 |
| column chromatography | | | |

Note:
The symbol “*” means α-isomaltosyl-transferring enzyme.

Experiment 4-5

### Property of α-isomaltosyl-transferring enzyme

The purified specimen of α-isomaltosyl-transferring enzyme in Experiment 4-4 was subjected to SDS-PAGE using a 7.5% (w/v) of polyacrylamide gel and then determined for molecular weight by comparing with the dynamics of standard molecular markers electrophoresed in parallel, commercialized by Bio-Rad Laboratories Inc., Brussels, Belgium, revealing that the enzyme had a molecular weight of about 102,000±20,000 daltons.

A fresh preparation of the above purified specimen was subjected to isoelectrophoresis using a gel containing 2% (w/v) ampholine commercialized by Amersham Corp., Div., Amersham International, Arlington Heights, Ill., USA, and then measured for pHs of protein bands and gel to determine the isoelectric point of the enzyme, revealing that the enzyme had an isoelectric point of about 5.6±0.5.

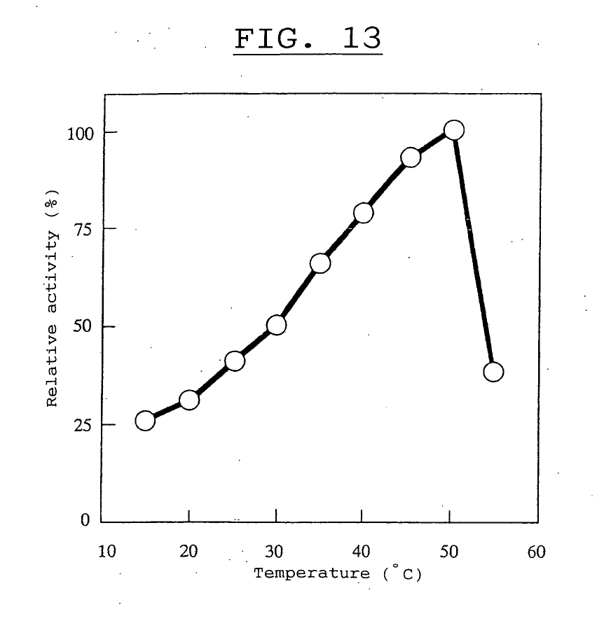

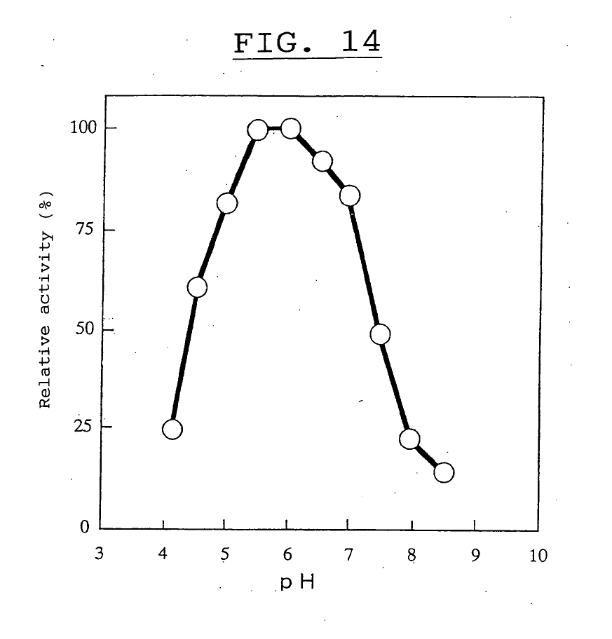

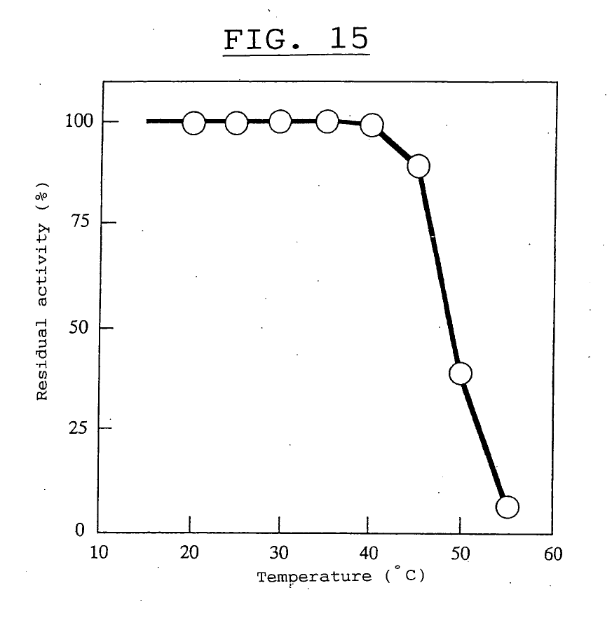

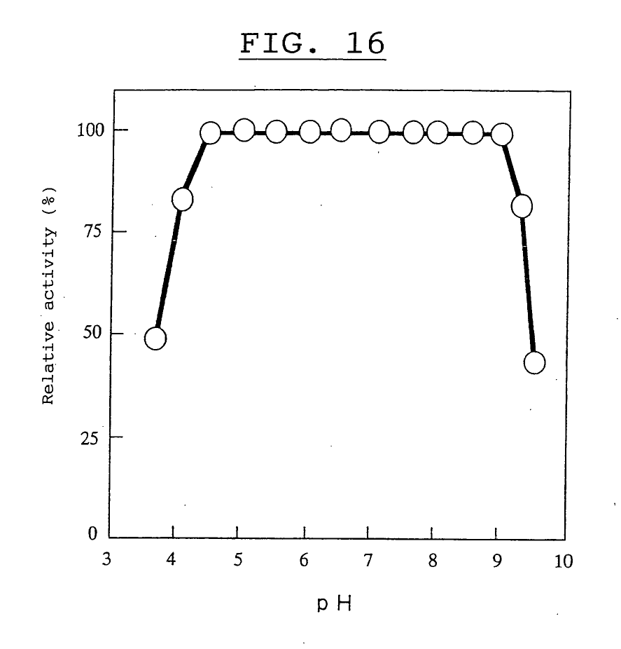

The influence of temperature and pH on the activity of α-isomaltosyl-transferring enzyme was examined in accordance with the assay for the enzyme activity. These results are in FIG. 13 (influence of temperature) and FIG. 14 (influence of pH). The optimum temperature of the enzyme was about 50° C. when incubated at pH 6.0 for 30 min. The optimum pH of the enzyme was about 5.5 to about 6.0 when incubated at 35° C. for 30 min. The thermal stability of the enzyme was determined by incubating the testing enzyme solutions in 20 mM acetate buffer (pH 6.0) at prescribed temperatures for 60 min, cooling with water the resulting enzyme solutions, and assaying the remaining enzyme activity of each solution. The pH stability of the enzyme was determined by keeping the testing enzyme solutions in 50 mM buffers having prescribed pHs at 4° C. for 24 hours, adjusting the pH of each solution to 6.0, and assaying the remaining enzyme activity of each solution. These results are respectively in FIG. 15 (thermal stability) and FIG. 16 (pH stability). As a result, the enzyme had thermal stability of up to about 40° C. and pH stability of about 4.5 to about 9.0.

The influence of metal ions on the activity of α-isomaltosyl-transferring enzyme was examined in the presence of 1 mM of each metal-ion according to the assay for the enzyme activity. The results are in Table 8.

TABLE 8

| | Relative activity | | Relative activity |
|---|---|---|---|
| Metal ion | (%) | Metal ion | (%) |
| None | 100 | Hg²⁺ | 2 |
| Zn²⁺ | 83 | Ba²⁺ | 90 |
| Mg²⁺ | 91 | Sr²⁺ | 93 |
| Ca²⁺ | 91 | Pb²⁺ | 74 |
| Co²⁺ | 89 | Fe²⁺ | 104 |
| Cu²⁺ | 56 | Fe³⁺ | 88 |
| Ni²⁺ | 89 | Mn²⁺ | 93 |
| Al³⁺ | 89 | EDTA | 98 |

As evident form the results in Table 8, the enzyme activity was greatly inhibited by Hg²⁺ and also inhibited by Cu²⁺. It was also found that the enzyme was not activated by Ca²⁺ and not inhibited by EDTA.

Experiment 4-6

### Amino acid sequence of α-isomaltosyl-transferring enzyme

The specification does not describe in detail the method for analyzing the amino acid sequence of α-isomaltosyl-transferring enzyme because it is disclosed in detail in Japanese Patent Application No. 350,142/00. Similarly as the polypeptide disclosed in the application, the α-isomaltosylglucosaccharide-forming enzyme in Experiment 4-4 has an amino acid sequence of the residues 30-1093 of SEQ ID NO:2 shown in parallel with nucleosides.

Experiment 5

### Action of α-isomaltosylglucosaccharide-forming enzyme on saccharides

The action of α-isomaltosylglucosaccharide-forming enzyme on saccharides as substrates was tested. First, a solution of maltose, maltotriose, maltotetraose, maltopentaose, maltohexaose, maltoheptaose, isomaltose, isomaltotriose, panose, isopanose, α,α-trehalose (may be abbreviated as “trehalose” hereinafter), kojibiose, nigerose, neotrehalose, cellobiose, gentibiose, maltitol, maltotriitol, lactose, sucrose, erlose, selaginose, maltosyl glucoside, or isomaltosyl glucoside was prepared. To each of the above solutions was added two units/g substrate of the purified specimen of α-isomaltosylglucosaccharide-forming enzyme from Bacillus globisporus C9 in Experiment 2-1 or Bacillus globisporus C11 in Experiment 4-1, and the resulting each solution was adjusted to give a substrate concentration of 2% (w/v) and incubated at 30° C. and pH 6.0 for 24 hours. The solutions before and after the enzymatic reactions were respectively subjected to thin-layer chromatography (abbreviated as “TLC” hereinafter). TLC was carried out in such a manner of separating saccharides by developing the solutions twice each using, as a developer, a mixture solution of n-butanol, pyridine, and water (=6:4:1), and, as a thin-layer plate, “KIESELGEL 60”, an aluminum plate (20×20 cm) for TLC commercialized by Merck & Co., Inc., Rahway, USA.; detecting the total saccharides in each mixture solution by spraying a mixture of sulfuric acid and methanol onto the aluminum plates to develop color of the total saccharides and detecting non-reducing saccharides in each mixture solution by the diphenylamine-aniline method. The results on TLC are in Table 9.

TABLE 9

Enzymatic action

| | Enzyme of | Enzyme of |
|---|---|---|
| Substrate | Strain C9 | Strain C11 |
| Maltose | + | + |
| Maltotriose | ++ | ++ |
| Maltotetraose | +++ | +++ |
| Maltopentaose | +++ | +++ |
| Maltohexaose | +++ | +++ |
| Maltoheptaose | +++ | +++ |
| Isomaltose | − | − |
| Isomaltotriose | − | − |
| Panose | − | − |
| Isopanose | ++ | ++ |
| Trehalose | − | − |
| Kojibiose | + | + |
| Nigerose | + | + |
| Neotrehalose | + | + |
| Cellobiose | − | − |
| Gentibiose | − | − |
| Maltitol | − | − |
| Maltotriitol | + | + |
| Lactose | − | − |
| Sucrose | − | − |
| Erlose | + | + |
| Selaginose | − | − |
| Maltosylglucoside | ++ | ++ |
| Isomaltosylglucoside | − | − |
| Note: | | |
| Before and after the enzymatic reaction, the symbols “−”, “+”, “++”, and “+++” mean that it showed no change, it showed a slight reduction of the color of substrate spot and the formation | | |
| # of other reaction product, it showed a high reduction of the color of substrate spot and the formation of other reaction product, and it showed a substantial disappearance of the color of substrate spot and the formation of other reaction product, respectively. | | |

As evident from the results in Table 9, it was revealed that the α-isomaltosylglucosaccharide-forming enzyme well acted on saccharides having both a glucose polymerization degree of at least three and a maltose structure at their non-reducing ends, among the saccharides tested. It was also found that the enzyme slightly acted on saccharides, having a glucose polymerization degree of two, such as maltose, kojibiose, nigerose, neotrehalose, maltotriitol, and erlose.

Experiment 6

### Reaction Product from Maltooligosaccharide

To an aqueous solution containing one percent (w/v) of maltose, maltotriose, maltotetraose, or maltopentaose as a substrate was added the purified specimen of α-isomaltosylglucosaccharide-forming enzyme in Experiment 4-1 in an amount of two units/g solid for the aqueous solution of maltose or maltotriose, 0.2 unit/g solid for that of maltotetraose, and 0.1 unit/g solid for that of maltopentaose, followed by incubation at 35° C. and pH 6.0 for eight hours. After a 10-min incubation at 100° C., the enzymatic reaction was suspended. The resulting reaction solutions were respectively measured for saccharide composition on HPLC using “YMC PACK ODS-AQ303”, a column commercialized by YMC Co., Ltd., Tokyo, Japan, at a column temperature of 40° C. and a flow rate of 0.5 ml/min of water, and using as a detector “RI-8012”, a differential refractometer commercialized by Tosoh Corporation, Tokyo, Japan. The results are in Table 10.

TABLE 10

| | Substrate | | | |
|---|---|---|---|---|
| Saccharide | | Malto- | Malto- | Malto- |
| as reaction product | Maltose | triose | tetraose | pentaose |
| Glucose | 8.5 | 0.1 | 0.0 | 0.0 |
| Maltose | 78.0 | 17.9 | 0.3 | 0.0 |
| Maltotriose | 0.8 | 45.3 | 22.7 | 1.9 |
| Maltotetraose | 0.0 | 1.8 | 35.1 | 19.2 |
| Maltopentaose | 0.0 | 0.0 | 3.5 | 34.4 |
| Maltohexaose | 0.0 | 0.0 | 0.0 | 4.6 |
| Isomaltose | 0.5 | 0.0 | 0.0 | 0.0 |
| Glucosylmaltose | 8.2 | 1.2 | 0.0 | 0.0 |
| Glucosylmaltotriose | 2.4 | 31.5 | 6.8 | 0.0 |
| X | 0.0 | 2.1 | 30.0 | 11.4 |
| Y | 0.0 | 0.0 | 1.4 | 26.8 |
| Z | 0.0 | 0.0 | 0.0 | 1.7 |
| Others | 0.6 | 0.1 | 0.2 | 0.0 |

Note:
In the table, glucosylmaltose means α-isomaltosylglucose, alias 6²-O-α-glucosylmaltose or panose; glucosylmaltotriose means α-isomaltosylmaltose, alias 6³-O-α-glucosylmaltotriose; X means the α-isomaltosylglucotriose in Experiment 7, alias 6⁴-O-α-glucosylmaltotetraose; Y means the α-isomaltosylglucotetraose in Experiment 7, alias 6⁵-O-α-glucosylmaltopentaose; and Z means an unidentified saccharide.

As evident from the results in Table 10, it was revealed that, after the action of the enzyme, glucose and α-isomaltosylglucose, alias 6²-O-α-glucosylmaltose or panose, were mainly formed and maltotriose, isomaltose, and α-isomaltosylmaltose, alias 6³-O-α-glucosylmaltotriose were formed in a small amount when the enzyme acted on maltose as a substrate. Also, it was revealed that, from maltotriose as a substrate, maltose and α-isomaltosylmaltose were mainly formed along with small amounts of glucose, maltotetraose, α-isomaltosylglucose alias 6²-O-α-glucosylmaltose or panose, and the product X. It was also found that, from maltotetraose as a substrate, maltotriose and the product X were mainly formed along with small amounts of maltose, maltopentaose, α-isomaltosylmaltose alias 6³-O-α-glucosylmaltotriose or panose, and the product Y. Further, it was revealed that, from maltopentaose as a substrate, maltotetraose and the product Y were mainly formed along with small amounts of maltotriose, maltohexaose, and the products X and Z.

The product X as a main product from maltotetraose as a substrate and the product Y as a main product from maltopentaose as a substrate were respectively isolated and purified as follows: The products X and Y were respectively purified on HPLC using “YMC PACK ODS-A R355-15S-15 12A”, a separatory HPLC column commercialized by YMC Co., Ltd., Tokyo, Japan, to isolate a specimen of the product X having a purity of at least 99.9% from the reaction product from maltotetraose in a yield of about 8.3%, d.s.b., and a specimen of the product Y having a purity of at least 99.9% from the reaction product from maltotetraose in a yield of about 11.5%, d.s.b.

Experiment 7

### Structural Analysis on Reaction Product

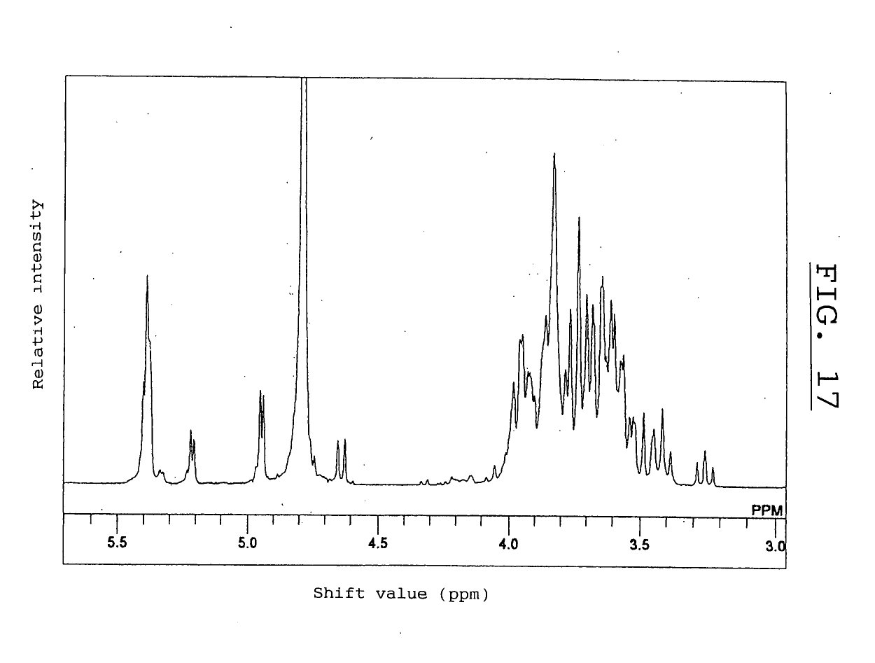

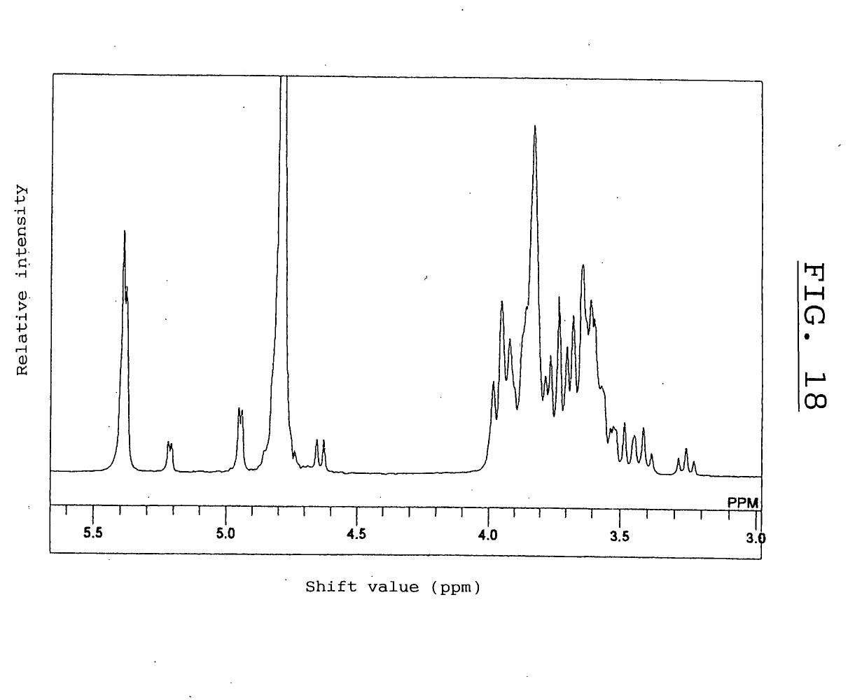

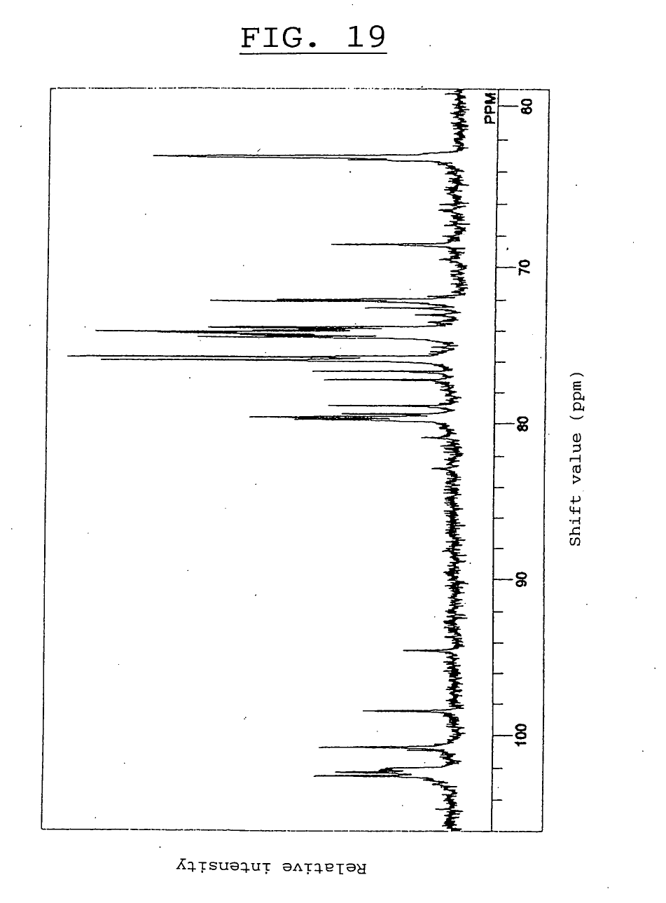

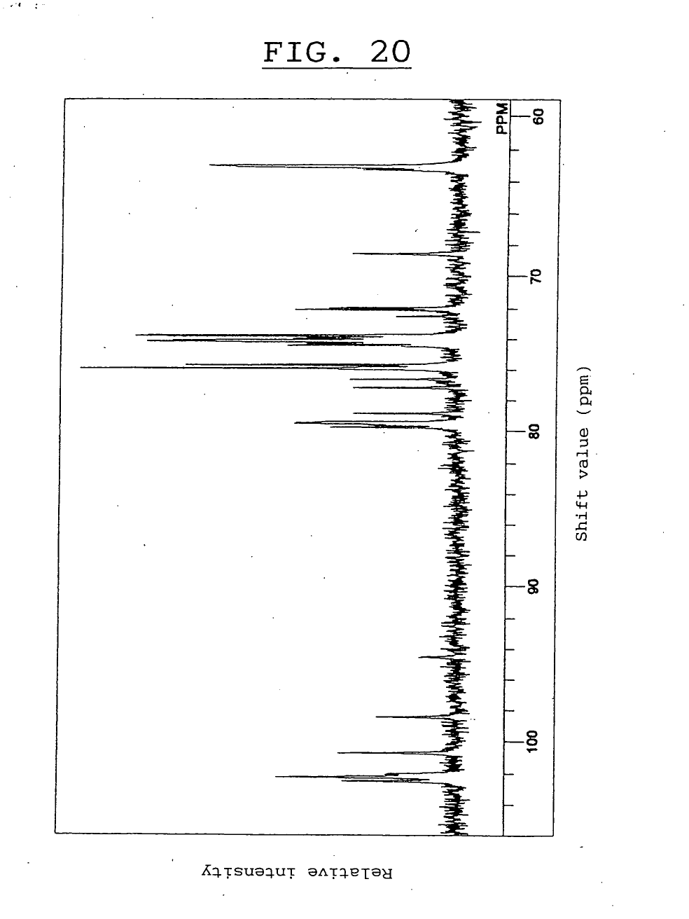

Using the products X and Y obtained by the method in Experiment 6, they were subjected to methyl analysis and NMR analysis in a usual manner. The results on their methyl analyses are in Table 11. For the results on their NMR analyses, FIG. 17 is a ¹H-NMR spectrum for the product X and FIG. 18 is for the product Y. The ¹³C-NMR spectra for the products X and Y are respectively in FIGS. 19 and 20, and their assignments are in Table 12.

TABLE 11

| | |
|---|---|
| Analyzed | Ratio |

| methyl compound | Product X | Product Y |
|---|---|---|
| 2,3,4-trimethyl compound | 1.00 | 1.00 |
| 2,3,6-trimethyl compound | 3.05 | 3.98 |
| 2,3,4,6-tetramethyl compound | 0.82 | 0.85 |

TABLE 12

| | Carbon | NMR chemical shift value (ppm) | |
|---|---|---|---|
| Glucose number | number | Product X | Product Y |
| a | 1 a | 100.8 | 100.8 |
| | 2 a | 74.2 | 74.2 |
| | 3 a | 75.8 | 75.7 |
| | 4 a | 72.2 | 72.2 |
| | 5 a | 74.5 | 74.5 |
| | 6 a | 63.2 | 63.1 |
| b | 1 b | 102.6 | 102.6 |
| | 2 b | 74.2 | 74.2 |
| | 3 b | 75.8 | 75.7 |
| | 4 b | 72.1 | 72.1 |
| | 5 b | 74.0 | 74.0 |
| | 6 b | 68.6 | 68.6 |
| c | 1 c | 102.3 | 102.3 |
| | 2 c | 74.2 | 74.2 |
| | 3 c | 76.0 | 76.0 |
| | 4 c | 79.6 | 79.5 |
| | 5 c | 73.9 | 73.9 |
| | 6 c | 63.2 | 63.1 |
| d | 1 d | 102.2 | 102.3 |
| | 2 d | 74.0 (α), 74.4 (β) | 74.2 |
| | 3 d | 76.0 | 76.0 |
| | 4 d | 79.8 | 79.5 |
| | 5 d | 73.9 | 73.9 |
| | 6 d | 63.2 | 63.1 |
| e | 1 e | 94.6 (α), 98.5 (β) | 102.1 |
| | 2 e | 74.2 (α), 76.7 (β) | 74.0 (α), 74.4 (β) |
| | 3 e | 75.9 (α), 78.9 (β) | 76.0 |
| | 4 e | 79.6 (α), 79.4 (β) | 79.8 |
| | 5 e | 72.6 (α), 77.2 (β) | 73.9 |
| | 6 e | 63.4 (α), 63.4 (β) | 63.1 |
| f | 1 f | | 94.6 (α), 98.5 (β) |
| | 2 f | | 74.2 (α), 76.7 (β) |
| | 3 f | | 76.0 (α), 78.9 (β) |
| | 4 f | | 79.6 (α), 79.5 (β) |
| | 5 f | | 72.6 (α), 77.2 (β) |
| | 6 f | | 63.3 (α), 63.3 (β) |

Based on these results, the product X formed from maltotetraose via the action of the α-isomaltosylglucosaccharide-forming enzyme was revealed as a pentasaccharide, in which a glucose residue binds via the α-linkage to OH-6 of glucose at the non-reducing end of maltotetraose, i.e., α-isomaltosylmaltotriose, alias 6⁴-O-α-glucosylmaltotetraose, represented by Formula 1.

Formula 1:
α-D-Glcp-(1→6)-α-D-Glcp-(1→4)-α-D-Glcp-(1→4)-α-D-
Glcp-(1→4)-D-Glcp

The product Y formed from maltopentaose was revealed as a hexasaccharide, in which a glucosyl residue binds via the α-linkage to OH-6 of glucose at the non-reducing end of maltopentaose, i.e., α-isomaltosylglucotetraose alias 6⁵-O-α-glucosylmaltopentaose, represented by Formula 2.

Formula 2:
α-D-Glcp-(1→6)-α-D-Glcp-(1→4)-α-D-Glcp-(1→4)-α-D-
Glcp-(1→4)-α-D-Glcp-(1→4)-D-Glcp

Based on these results, it was concluded that the α-isomaltosylglucosaccharide-forming enzyme acts on maltooligosaccharides as shown below:

(1) The enzyme acts on as substrates maltooligosaccharides having a glucose polymerization degree of at least two where glucoses are linked together via the α-1,4 linkage, and catalyzes the intermolecular 6-glucosyl-transferring reaction in such a manner of transferring a glucosyl residue at the non-reducing end of a maltooligosaccharide molecule to C-6 of the non-reducing end of other maltooligosaccharide molecule to form both an α-isomaltosylglucosaccharide alias 6-O-α-glucosylmaltooligosaccharide, having a 6-O-α-glucosyl residue and a higher glucose polymerization degree by one as compared with the intact substrate, and a maltooligosaccharide with a reduced glucose polymerization degree by one as compared with the intact substrate; and

(2) The enzyme slightly catalyzes the 4-glucosyl-transferring reaction and forms both a maltooligosaccharide, having an increased glucose polymerization degree by one as compared with the intact substrate, and a maltooligosaccharide having a reduced glucose polymerization degree by one as compared with the intact substrate.

Experiment 8

### Specificity of Saccharide Transferring Reaction Acceptor

Using different saccharides, it was tested whether the saccharides were used as saccharide transferring reaction acceptors for the α-isomaltosylglucosaccharide-forming enzyme. A 1.6% solution, as a solution of saccharide transferring reaction acceptor, of D-glucose, D-xylose, L-xylose, D-galactose, D-fructose, D-mannose, D-arabinose, D-fucose, L-sorbose, L-rhamnose, methyl-α-glucopyranoside (methyl-α-glucose), methyl-β-glucopyranoside (methyl-β-glucose), N-acetyl-glucosamine, sorbitol, α,α-trehalose, isomaltose, isomaltotriose, cellobiose, gentibiose, maltitol, lactose, sucrose, α-cyclodextrin, β-cyclodextrin, or γ-cyclodextrin, was prepared. To each solution with a saccharide concentration was added “PINE-DEX #100”, a partial starch hydrolysate, as a saccharide donor, to give a concentration of 4%, and admixed with one unit/g saccharide donor, d.s.b., of either of purified specimens of α-isomaltosylglucosaccharide-forming enzyme from Bacillus globisporus C9 strain obtained by the method in Experiment 2-1, Bacillus globisporus C11 strain obtained by the method in Experiment 4-1. The resulting mixture solutions were incubated at 30° C. and pH 6.0 except that the enzyme from Arthrobacter globiformis A19 strain was incubated at pH 8.4 for 24 hours. The reaction mixtures of the post-enzymatic reactions were analyzed on gas chromatography (abbreviated as “GLC” hereinafter) for monosaccharides and disaccharides as acceptors, and on HPLC for trisaccharides as acceptors to confirm whether these saccharides could be used as their saccharide transferring reaction acceptors. In the case of performing GLC, the following apparatuses and conditions are used: GLC apparatus, “GC-16A” commercialized by Shimadzu Corporation, Tokyo, Japan; column, a stainless-steel column, 3 mm in diameter and 2 m in length, packed with 2% “SILICONE OV-17/CHROMOSOLV W”, commercialized by GL Sciences Inc., Tokyo, Japan; carrier gas, nitrogen gas at a flow rate of 40 ml/min under temperature conditions of increasing from 160° C. to 320° C. at an increasing temperature rate of 7.5° C./min; and detection, a hydrogen flame ionization detector. In the case of HPLC analysis, the apparatuses and conditions used were: HPLC apparatus, “CCPD” commercialized by Tosoh Corporation, Tokyo, Japan; column, “ODS-AQ-303” commercialized by YMC Co., Ltd., Tokyo, Japan; eluent, water at a flow rate of 0.5 ml/min; and detection, a differential refractometer. The results are in Table 13.

TABLE 13

Product of saccharide
transferring reaction

| | Enzyme of | Enzyme of |
|---|---|---|
| Saccharide | Strain C9 | Strain C11 |
| D-Glucose | + | + |
| D-Xylose | ++ | ++ |
| L-Xylose | ++ | ++ |
| D-Galactose | + | + |
| D-Fructose | + | + |
| D-Mannose | − | − |
| D-Arabinose | ± | ± |
| D-Fucose | + | + |
| L-Sorbose | + | + |
| L-Rhamnose | − | − |
| Methyl-α- | ++ | ++ |
| glucopyranoside | | |
| Methyl-β- | ++ | ++ |
| glucopyranoside | | |
| N-Acetyl- | + | + |
| glucosamine | | |
| Sorbitol | − | − |
| Trehalose | ++ | ++ |
| Isomaltose | ++ | ++ |
| Isomaltotriose | ++ | ++ |
| Cellobiose | ++ | ++ |
| Gentibiose | ++ | ++ |
| Maltitol | ++ | ++ |
| Lactose | ++ | ++ |
| Sucrose | ++ | ++ |
| α-Cyclodextrin | − | − |
| β-Cyclodextrin | − | − |
| γ-Cyclodextrin | − | − |
| Note: | | |
| In the table, the symbols “−”, “±”, “+”, and “++” mean that no saccharide-transferred product was detected through transferring reaction to acceptor; a saccharide-transferred product was detected in an amount of less than one percent | | |
| # through transfer reaction to acceptor; a saccharide-transferred product was detected in an amount of at least one percent but less than ten percent through transferring reaction to acceptor; and a saccharide-transferred product was detected in an amount of at least ten percent through transferring reaction to acceptor. | | |

As evident from the results in Table 13, α-isomaltosylglucosaccharide-forming enzyme utilizes different types of saccharides as saccharide transfer acceptors, particularly, the enzyme has a higher saccharide transferring action, particularly, on D-/L-xylose, methyl-α-glucopyranoside, methyl-β-glucopyranoside, α,α-trehalose, isomaltose, isomaltotriose, cellobiose, gentibiose, maltitol, lactose, and sucrose; then on D-glucose, D-fructose, D-fucose, L-sorbose, and N-acetylglucosamine, as well as D-arabinose.

Experiment 9

### Preparation of Cyclotetrasaccharide from Culture

A liquid medium consisting of 5% (w/v) of “PINE-DEX #1”, a partial starch hydrolysate commercialized by Matsutani Chemical Ind., Tokyo, Japan, 1.5% (w/v) of “ASAHIMEAST”, a yeast extract commercialized by Asahi Breweries, Ltd., Tokyo, Japan, 0.1% (w/v) of dipotassium phosphate, 0.06% (w/v) of sodium phosphate dodecahydrate, 0.05% (w/v) magnesium sulfate heptahydrate, and water was placed in a 500-ml Erlenmeyer flask in an amount of 100 ml, sterilized by autoclaving at 121° C. for 20 min, cooled, and then seeded with Bacillus globisporus C9 strain, FERM BP-7143, followed by culturing under rotary-shaking conditions at 27° C. and 230 rpm for 48 hours and centrifuging the resulting culture to remove cells to obtain a supernatant. The supernatant was autoclaved at 120° C. for 15 min and then cooled, and the resulting insoluble substances were removed by centrifugation to obtain a supernatant. About 90 ml of the supernatant was adjusted to pH 5.0 and 45° C. and then incubated for 24 hours after admixed with 1,500 units per gram of solids of “TRANSGLUCOSIDASE L AMANO™”, an α-glucosidase commercialized by Amano Pharmaceutical Co., Ltd., Aichi, Japan, and 75 units per gram of solids of a glucoamylase commercialized by Nagase Biochemicals, Ltd., Kyoto, Japan. Thereafter, the resulting culture was adjusted to pH 12 by the addition of sodium hydroxide and boiled for two hours to decompose the remaining reducing sugars. After removing insoluble substances by filtration, the resulting solution was decolored and desalted with “DIAION PK218” and “DIAION WA30”, cation exchange resins commercialized by Mitsubishi Chemical Industries, Ltd., Tokyo, Japan, and further desalted with “DIAION SK-1B”, commercialized by Mitsubishi Chemical Industries, Ltd., Tokyo, Japan, and “AMBERLITE IRA411”, an anion exchange resin commercialized by Japan Organo Co., Ltd., Tokyo, Japan, followed by decoloring with an activated charcoal, membrane filtered, concentrated by an evaporator, and lyophilized in vacuo to obtain about 0.6 g, d.s.b., of a saccharide powder with a cyclotetrasaccharide content of 99.9% or higher.

Experiment 10

### Formation of Cyclotetrasaccharide

The formation test on cyclotetrasaccharide by the action of α-isomaltosylglucosaccharide-forming enzyme and α-isomaltosyl-transferring enzyme was conducted using saccharides. Using as saccharides maltose, maltotriose, maltotetraose, maltopentaose, amylose, soluble starch, “PINE-DEX #100”, a partial starch hydrolyzate commercialized by Matsutani Chemical Ind., Tokyo, Japan, or glycogen from oyster commercialized by Wako Pure Chemical Industries Ltd., Tokyo, Japan, solutions containing each of the saccharides were respectively prepared.

To each of these solutions with a respective concentration of 0.5%, one unit/g solid of a purified specimen of α-isomaltosylglucosaccharide-forming enzyme from Strain C11 obtained by the method in Experiment 4-1 and 10 units/g solid of a purified specimen of α-isomaltosyl-transferring enzyme from Strain C11 obtained by the method in Experiment 4-4, and the resulting mixture was subjected to an enzymatic reaction at 30° C. and pH 6.0. The enzymatic conditions were the following four systems:

(1) After the α-isomaltosylglucosaccharide-forming enzyme was allowed to act on a saccharide solution for 24 hours, the enzyme was inactivated by heating, and then the α-isomaltosyl-transferring enzyme was allowed to act on the resulting mixture for 24 hours and inactivated by heating;

(2) After the α-isomaltosylglucosaccharide-forming enzyme and the α-isomaltosyl-transferring enzyme were allowed in combination to act on a saccharide solution for 24 hours, then the saccharides were inactivated by heating;

(3) After only the α-isomaltosylgluco-saccharide-forming enzyme was allowed to act on a saccharide solution for 24 hours, then the enzyme was inactivated by heating; and

(4) After only the α-isomaltosyl-transferring enzyme was allowed to act on a saccharide solution for 24 hours, then the enzyme was inactivated by heating.

To determine the formation level of cyclotetra-saccharide in each reaction mixture after heating, the reaction mixture was treated with α-glucosidase and glucoamylase similarly as in Experiment 1 to hydrolyze the remaining reducing oligosaccharides, followed by quantitation of cyclotetrasaccharide on HPLC. The results are in Table 14.

TABLE 14

| | Formation yield of cyclotetrasaccharide (%) | | | |
|---|---|---|---|---|
| Substrate | A | B | C | D |
| Maltose | 4.0 | 4.2 | 0.0 | 0.0 |
| Maltotriose | 10.2 | 12.4 | 0.0 | 0.0 |
| Maltotetraose | 11.3 | 21.5 | 0.0 | 0.0 |
| Maltopentaose | 10.5 | 37.8 | 0.0 | 0.0 |
| Amylose | 3.5 | 31.6 | 0.0 | 0.0 |
| Soluble starch | 5.1 | 38.2 | 0.0 | 0.0 |
| Partial starch | 6.8 | 63.7 | 0.0 | 0.0 |
| hydrolyzate | | | | |
| Glycogen | 10.2 | 86.9 | 0.0 | 0.0 |
| Note: | | | | |
| The symbols “A”, “B”, “C” and “D” mean that α-isomaltosylglucosaccharide-forming enzyme was first allowed to act on a substrate and then α-isomaltosyl-transferring enzyme was allowed to act on the resulting mixture, the | | | | |
| # α-isomaltosylglucosaccharide-forming enzyme and α-isomaltosyl-transferring enzyme were allowed to coact on a substrate, only α-isomaltosylglucosaccharide-forming enzyme was allowed to act on a substrate, and only α-isomaltosyl-transferring enzyme was allowed to act on a substrate. | | | | |

As evident from the results in Table 14, no cyclotetrasaccharide was formed from any of the saccharides tested by the action of only α-isomaltosylglucosaccharide-forming enzyme or α-isomaltosyl-transferring enzyme, but cyclotetrasaccharide was formed by the coaction of these enzymes. It was revealed that the formation level of cyclotetrasaccharide was relatively low as below about 11% when α-isomaltosyl-transferring enzyme was allowed to act on the substrate saccharides after the action of α-isomaltosylglucosaccharide-forming enzyme, while the formation level was increased by simultaneously allowing the enzymes to act on every saccharide tested, particularly, increased to about 87% and about 64% when the enzymes were allowed to act on glycogen and partial starch hydrolyzate, respectively.

Based on the reaction properties of α-isomaltosylglucosaccharide-forming enzyme and α-isomaltosyl-transferring enzyme, the formation mechanism of cyclotetrasaccharide by the coaction of these enzymes is estimated as follows:

(1) α-Isomaltosylglucosaccharide-forming enzyme acts on a glucose residue at the non-reducing end of an α-1,4 glucan chain of glycogen and partial starch hydrolyzates, etc., and intermolecularly transfers the glucose residue to OH-6 of a glucose residue at the non-reducing end of other α-1,4 glucan chain of glycogen to form an α-1,4 glucan chain having an α-isomaltosyl residue at the non-reducing end;

(2) α-Isomaltosyl-transferring enzyme acts on the α-1,4 glucan chain having an α-isomaltosyl residue at the non-reducing end and intermolecularly transfers the isomaltosyl residue to C-3 of glucose residue at the non-reducing end of other α-1,4 glucan chain having isomaltosyl residue at the non-reducing end to form an α-1,4 glucan chain having an isomaltosyl-1,3-isomaltosyl residue at the non-reducing end;

(3) Then, α-isomaltosyl-transferring enzyme acts on the α-1,4 glucan chain having an isomaltosyl-1,3-isomaltosyl residue at the non-reducing end and releases the isomaltosyl-1,3-isomaltosyl residue from the α-1,4 glucan chain via the intramolecular transferring reaction to cyclize the released isomaltosyl-1,3-isomaltosyl residue into cyclotetra-saccharide;

(4) From the released α-1,4 glucan chain, cyclotetrasaccharide is newly formed through the sequential steps (1) to (3). Thus, it is estimated that the coaction of α-isomaltosylglucosaccharide-forming enzyme and α-isomaltosyl-transferring enzyme increases the formation of cyclotetra-saccharide in the above sequential manner.

Experiment 11

### Influence of Liquefaction Degree of Starch

A 15% corn starch suspension was prepared, admixed with 0.1% calcium carbonate, adjusted to pH 6.0, and then mixed with 0.2-2.0% per gram starch of “TERMAMYL 60L”, an α-amylase specimen commercialized by Novo Indutri A/S, Copenhagen, Denmark, followed by the enzymatic reaction at 95° C. for 10 min. Thereafter, the reaction mixture was autoclaved at 120° C. for 20 min, promptly cooled to about 35° C. to obtain a liquefied starch with a DE (dextrose equivalent) of 3.2-20.5. To the liquefied starch were added two units/g solid of a purified specimen of α-isomaltosylglucosaccharide-forming enzyme from Strain C11 obtained by the method in Experiment 4-1, and 20 units/g solid of a purified specimen of α-isomaltosyl-transferring enzyme from Strain C11 obtained by the method in Experiment 4-4, followed by the incubation at 35° C. for 24 hours. After completion of the reaction, the reaction mixture was heated at 100° C. for 15 min to inactivate the remaining enzymes. Then, the reaction mixture thus obtained was treated with α-glucosidase and glucoamylase similarly as in Experiment 1 to hydrolyze the remaining reducing oligosaccharides, followed by quantifying the formed cyclotetrasaccharide on HPLC. The results are in Tale 15.

TABLE 15

| Amount of α-amylase | | Yield of |
|---|---|---|
| per starch (%) | DE | cyclotetrasaccharide (%) |
| 0.2 | 3.2 | 54.5 |
| 0.4 | 4.8 | 50.5 |
| 0.6 | 7.8 | 44.1 |
| 1.0 | 12.5 | 39.8 |
| 1.5 | 17.3 | 34.4 |
| 2.0 | 20.5 | 30.8 |

As evident from the results in Table 15, it was revealed that the formation of cyclotetrasaccharide by the coaction of α-isomaltosylglucosaccharide-forming enzyme and α-isomaltosyl-transferring enzyme is influenced by the liquefaction degree of starch, i.e., the lower the liquefaction degree or the lower the DE, the more the yield of cyclotetrasaccharide from starch becomes. On the contrary, the higher the liquefaction degree or the high the DE, the lower the yield of cyclotetrasaccharide from starch becomes. It was revealed that a suitable liquefaction degree is a DE of about 20 or lower, preferably, DE of about 12 or lower, more preferably, DE of about five or lower.

Experiment 12

### Influence of Concentration of Partial Starch Hydrolyzate

Aqueous solutions of “PINE-DEX #100”, a partial starch hydrolyzate with a DE of about two to about five, having a final concentration of 0.5-40%, were prepared and respectively admixed with one unit/g solid of a purified specimen of α-isomaltosylglucosaccharide-forming enzyme from Strain C11 obtained by the method in Experiment 4-1 and 10 units/g solid of a purified specimen of α-isomaltosyl-transferring enzyme from Strain C11 obtained by the method in Experiment 4-4, followed by the coaction of the enzymes at 30° C. and pH 6.0 for 48 hours. After completion of the reaction, the reaction mixture was heated at 100° C. for 15 min to inactivate the remaining enzymes, and then treated with α-glucosidase and glucoamylase similarly as in Experiment 1 to hydrolyze the remaining reducing oligosaccharides, followed by quantifying the formed cyclotetrasaccharide on HPLC. The results are in Table 16.

TABLE 16

| Concentration of | Formation yield of |
|---|---|
| PINE-DEX (%) | cyclotetrasaccharide (%) |
| 0.5 | 63.6 |
| 2.5 | 62.0 |
| 5 | 60.4 |
| 10 | 57.3 |
| 15 | 54.6 |
| 20 | 51.3 |
| 30 | 45.9 |
| 40 | 35.9 |

As evident from the results in Table 16, the formation yield of cyclotetrasaccharide was about 64% at a low concentration of 0.5%, while it was about 40% at a high concentration of 40%. The fact indicated that the formation yield of cyclotetrasaccharide increased depending on the concentration of partial starch hydrolyzate as a substrate. The result revealed that the formation yield of cyclotetrasaccharide increased as the decrease of partial starch hydrolyzate.

Experiment 13

### Influence of the Addition of Cyclodextrin Glucanotransferase

A 15% aqueous solution of “PINE-DEX #100”, a partial starch hydrolyzate was prepared and admixed with one unit/g solid of a purified specimen of α-isomaltosylglucosaccharide-forming enzyme from Strain C11 obtained by the method in Experiment 4-1, 10 units/g solid of a purified specimen of α-isomaltosyl-transferring enzyme from Strain C11 obtained by the method in Experiment 4-4, and 0-0.5 unit/g solid of cyclodextrin glucanotransferase (CGTase) from a microorganism of the species Bacillus stearothermophilus, followed by the coaction of these enzymes at 30° C. and pH 6.0 for 48 hours. After completion of the reaction, the reaction mixture was heated at 100° C. for 15 min to inactivate the remaining enzymes, and then treated with “TRANSGLUCOSIDASE L AMANO™”, an α-glucosidase commercialized by Amano Pharmaceutical Co., Ltd., Aichi, Japan, and a glucoamylase specimen commercialized by Nagase Biochemicals, Ltd., Kyoto, Japan, to hydrolyze the remaining reducing oligosaccharides, followed by quantifying the formed cyclotetrasaccharide on HPLC. The results are in Table 17.

TABLE 17

| Amount of CGTase added | Formation yield of |
|---|---|
| (unit) | cyclotetrasaccharide (%) |
| 0 | 54.6 |
| 2.5 | 60.1 |
| 5 | 63.1 |
| 10 | 65.2 |

As evident from the results in Table 17, it was revealed that the addition of CGTase increased the formation yield of cyclotetrasaccharide.

Experiment 15

### Preparation of Isomaltose-Releasing Enzyme

A liquid nutrient culture medium, consisting of 3.0% (w/v) of dextran, 0.7% (w/v) of peptone, 0.2% (w/v) of dipotassium phosphate, 0.05% (w/v) magnesium sulfate heptahydrate, and water was placed in 500-ml Erlenmeyer flasks in a volume of 100 ml each, autoclaved at 121° C. for 20 minutes to effect sterilization, cooled, inoculated with a stock culture of Arthrobacter globiformis, IAM 12103, and incubated at 27° C. for 48 hours under rotary shaking conditions of 230 rpm for use as a seed culture. About 20 L of a fresh preparation of the same nutrient culture medium as used in the above culture were placed in a 30-L fermentor, sterilized by heating, cooled to 27° C., inoculated with 1% (v/v) of the seed culture, and incubated for about 72 hours while stirring under aeration agitation conditions at 27° C. and a pH of 6.0-8.0. The resultant culture, having an activity of about 16.5 units/ml of α-isomaltodextranase as an isomaltose-releasing enzyme, was centrifuged at 10,000 rpm for 30 min to obtain about 18 L of a supernatant having an activity of about 16 units/ml of the enzyme and a total enzyme activity of about 288,000 units. The activity of isomaltodextranase was assayed as follows: Provide as a substrate solution a 1.25% (w/v) aqueous dextran solution containing 0.1M acetate buffer (pH 5.5), add one milliliter of an enzyme solution to the substrate solution, react the mixture solution at 40° C. for 20 min, collect one milliliter of the reaction mixture, added the collected reaction mixture to two milliliters of Somogyi reagent to suspend the enzymatic reaction, and quantify the reducing power of the formed isomaltose by the Somogyi-Nelson's method. One unit of isomaltodextranase activity was defined as the enzyme amount that exhibits a reducing power corresponding to that of one micromole of isomaltose per minute under the above enzymatic reaction conditions. About 18 L of the resulting supernatant was concentrated with a UF membrane into an about two liter solution which was then dialyzed against 80% saturated ammonium sulfate solution at 4° C. for 24 hours. The salted out precipitates were collected by centrifugation at 10,000 rpm for 30 min and dissolved in 5 mM phosphate buffer (pH 6.8), followed by dialyzing the resulting solution against a fresh preparation of the same phosphate buffer to obtain about 400 ml of a crude enzyme solution. The crude enzyme solution was subjected to ion-exchange chromatography using two liters of “SEPABEADS FP-DA13” gel. Isomaltodextranase was eluted in non-adsorbed fractions without adsorbing on the gel. The fractions with isomaltodextranase activity were collected, pooled and dialyzed against 80% saturated ammonium solution at 4° C. for 24 hours. The resulting precipitates were collected by centrifugation at 10,000 rpm for 30 min and dissolved in 5 mM phosphate buffer (pH 6.8), and the solution was dialyzed against a fresh preparation of the same phosphate buffer to obtain about 500 ml of a partially purified enzyme solution having an activity of 161,000 units of isomaltodextranase.

Experiment 16

### Preparation of Isomaltose from α-isomaltosylglucosaccharide and Cyclotetrasaccharide

To an aqueous solution having a final solid concentration of 0.2% (w/v) of panose, α-isomaltosylmaltose, α-isomaltosyltriose, α-isomaltosyltetraose, or cyclotetrasaccharide, were added 100 units/g solid of an isomaltodextranase specimen, obtained by the method in Experiment 15, except for using 100 or 3,000 units of the specimen for the aqueous solution of cyclotetrasaccharide, allowed to react at 40° C. and pH 5.5 for 24 hours, and kept at 100° C. for 20 min to suspend the enzymatic reactions. The saccharide composition for each reaction mixture was determined on HPLC. The conditions used in HPLC were: Column, “MCIGEL CK04SS” comercialized by Mitsubishi Chemical Industries, Ltd., Tokyo, Japan; 80° C., inner column temperature; 0.5 ml/min, a flow rate of water as an eluent; and detection, “RI-8012”, a diffraction refractometer commercialized by Tosoh Corporation, Tokyo, Japan. The results are in Table 18.

TABLE 18

| | Amount of | Saccharide formed | | | | | |
|---|---|---|---|---|---|---|---|
| | enzyme | (peak area (%) on HPLC) | | | | | |
| Substrate | (unit) | G1 | IM | G2 | G3 | G4 | A |
| IMG1 | 100 | 35 | 65 | 0 | 0 | 0 | 0 |
| IMG2 | 100 | 0 | 51 | 49 | 0 | 0 | 0 |
| IMG3 | 100 | 0 | 41 | 0 | 59 | 0 | 0 |
| IMG4 | 100 | 0 | 35 | 0 | 0 | 65 | 0 |
| Cyclotetra- | 100 | 0 | 22 | 0 | 0 | 0 | 78 |
| saccharide | 3,000 | 0 | 100 | 0 | 0 | 0 | 0 |
| Note: | | | | | | | |
| The symbols “IMG1”, “IMG2”, “IMG3” and “IMG4” mean panose, α-isomaltosylmaltose, α-isomaltosyltriose, and isomaltosyltetraose, respectively; the symbols “G1”, “IM”, “G2”, | | | | | | | |
| # “G3”, and “G4” mean glucose, isomaltose, maltose, maltotriose, and maltoteraose, respectively; and the symbol “A” means an intermediate product formed during the formation of isomaltose from cyclotetrasaccharide. | | | | | | | |

As evident from the result in Table 18, it was revealed that, when acts on α-isomaltosylglucosaccharides, isomaltodextranase forms only glucose and isomaltose from panose as a substrate; only isomaltose and maltose from α-isomaltosylmaltose as a substrate; only isomaltose and maltotriose from α-isomaltosyltriose as a substrate; and forms only isomaltose and maltotetraose from α-isomaltosyltetraose as a substrate. While it was revealed that the enzyme forms only isomaltose from cyclotetrasaccharide as a substrate through the product A as the intermediate.

Thereafter, the product A, as an intermediate formed from cyclotetrasaccharide as a substrate, was purified and isolated as follows: Using “YMC-PACK ODS-A R355-15S-15 12A”, a separatory HPLC column commercialized by YMC Co., Ltd., Tokyo, Japan, the product A was purified and isolated, resulting in an isolation of the product A, having a purity of at least 98.2% in a yield of about 7.2%, from the reaction products formed from the material cyclotetrasaccharide.

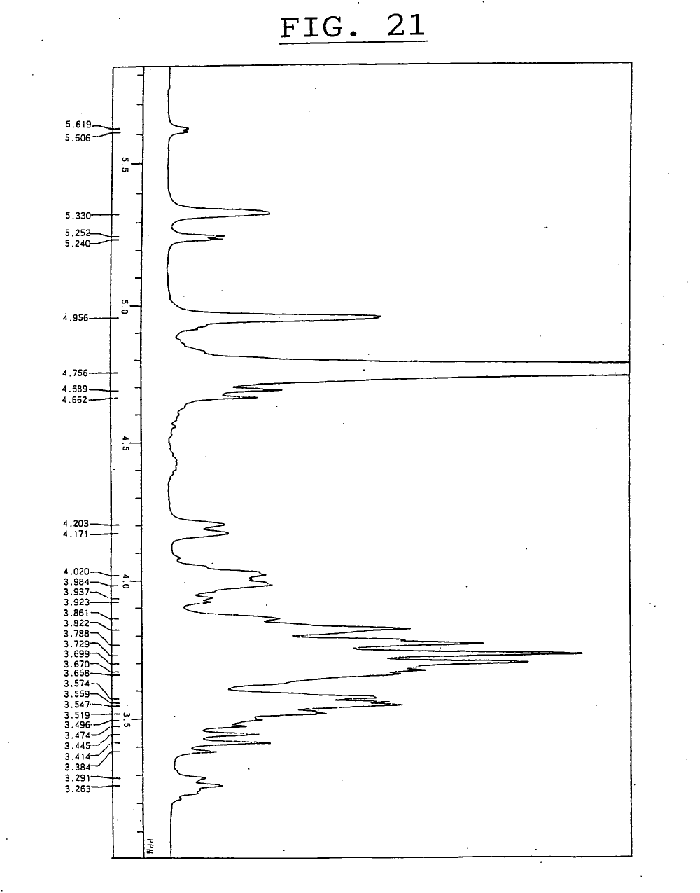

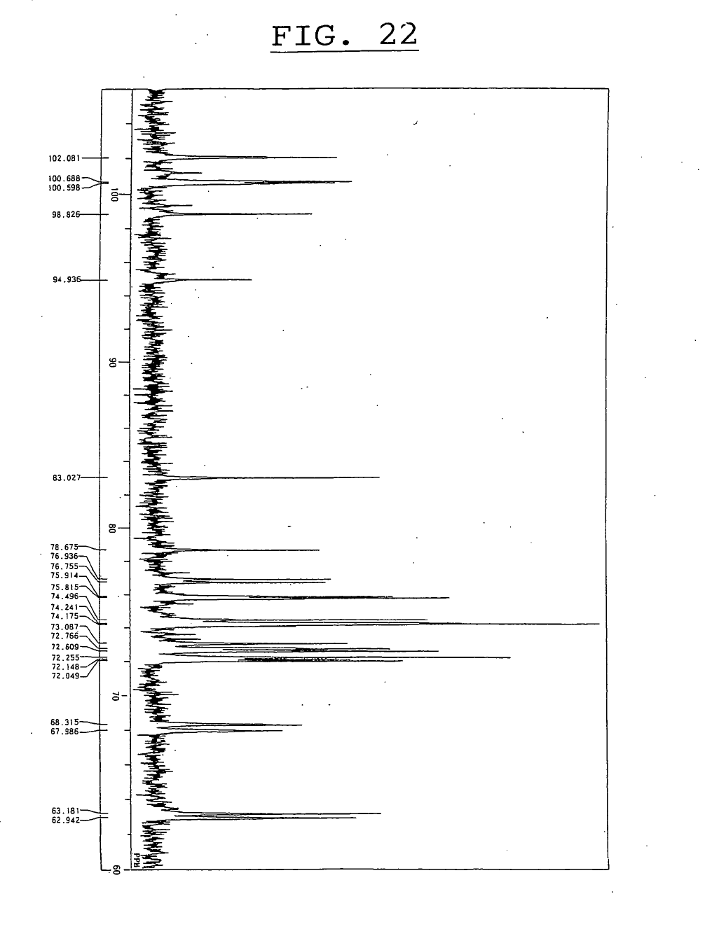

Upon the product A, it was subjected to methyl analysis and NMR analysis in a usual manner. The results on the methyl analysis is in Table 19. For the result on the NMR analysis, the ¹H-NMR spectrum is FIG. 21. The ¹³C-NMR spectrum for the product A is in FIG. 22, and the assignment thereof is tabulated in Table 20.

TABLE 19

| Analyzed | |
|---|---|
| methyl compound | Ratio |
| 2,3,4-trimethyl compound | 2.00 |
| 2,3,6-trimethy1 compound | 0.92 |
| 2,3,4,6-tetramethyl compound | 0.88 |

TABLE 20

| Glucose number | Carbon Number | NMR Chemical shift (ppm) |
|---|---|---|
| | 1 a | 100.7 |
| | 2 a | 74.2 |
| a | 3 a | 75.2 |
| | 4 a | 72.3 |
| | 5 a | 74.5 |
| | 6 a | 63.2 |
| | 1 b | 102.1 |
| | 2 b | 74.3 |
| b | 3 b | 75.9 |
| | 4 b | 72.6 |
| | 5 b | 74.2 |
| | 6 b | 68.0 |
| | 1 c | 100.6 |
| | 2 c | 72.8 |
| c | 3 c | 83.0 |
| | 4 c | 72.0 |
| | 5 c | 73.1 |
| | 6 c | 62.9 |
| | 1 e | 94.9 (α), 98.8 (β) |
| | 2 e | 74.1 (α), 76.6 (β) |
| e | 3 e | 75.8 (α), 78.7 (β) |
| | 4 e | 72.1 (α), 72.1 (β) |
| | 5 e | 72.6 (α), 76.9 (β) |
| | 6 e | 68.3 (α), 68.3 (β) |

Based on these results, it was revealed that the product A, as an intermediate, formed during the formation of isomaltose from cyclotetrasaccharide via the action of isomaltodextranase was a tetrasaccharide represented by Formula 3, α-D-glucosyl-(1→6)-α-D-glucosyl-(1→3)-α-D-glucosyl-(1→6)-α-D-glucose (designated as “ring-opened tetrasaccharide” hereinafter), obtained by hydrolyzing either of the α-1,3 linkages in cyclotetrasaccharide for ring opening. Formula 3:

α-D-Glcp-(1→6)-α-D-Glcp-(1→3)-α-D-Glcp-(1→6)-α-D-Glcp

Based on these results, the action of isomaltodextranase on α-isomaltosylglucosaccharide is judged as follows:

Isomaltodextranase acts on α-isomaltosyl-glucosaccharides having a 6-O-α-glucosyl residue as substrates to specifically hydrolyze the α-1,4 linkage between the isomaltosyl residue at the non-reducing end and the glucose residue (or a maltooligosaccharide residue) to form isomaltose and glucose (or a maltooligosaccharide). The enzyme also acts on cyclotetrasaccharide as a substrate and hydrolyzes its α-1,3 linkage, and further acts on a ring-opened tetrasaccharide and hydrolyzes its α-1,3 linkage to form isomaltose.

Experiment 17

### Formation of Isomaltose from Substrates

Using different substrates, the isomaltose formation by the action of α-isomaltosylglucosaccharide-forming enzyme and isomaltodextranase was tested. Using calcium chloride and maltose, maltotriose, maltotetraose, maltopentaose, maltohexaose, maltoheptaose, amylose, or “PINE-DEX #100”, a partial starch hydrolyzate commercialized by Matsutani Chemical Ind., Tokyo, Japan, aqueous solutions for saccharides each were prepared to give a final saccharide concentration of 5% and a final calcium chloride concentration of 1 mM. Then, to each solution were added 0.2 unit/g solids of the purified α-isomaltosylglucosaccharide-forming enzyme from Strain C11 obtained in Experiment 4-1 and 100 units/g solids of the isomaltodextranase obtained in Experiment 15, followed by reacting at 40° C. at pH 5.5. The reaction conditions were conducted in the following two reaction systems:

(1) After contacting α-isomaltosylglucosaccharide-forming enzyme with each saccharide for 65 hours, the enzyme was inactivated by heating, and then isomaltodextranase was further allowed to act on the saccharide for 65 hours and inactivated by heating.

(2) After contacting α-isomaltosylglucosaccharide-forming enzyme in combination with isomaltodextranase with each saccharide for 65 hours, the enzymes were inactivated by heating.

After the above enzymatic reactions, the formation yield of isomaltose in the resulting reaction mixtures received with heat treatment was quantified on HPLC. The results are in Table 21.

TABLE 21

Formation yield of isomaltose (%)

| Substrate | A | B |
|---|---|---|
| Maltose | 6.6 | 7.0 |
| Maltotriose | 15.7 | 18.7 |
| Maltotetraose | 15.8 | 45.4 |
| Maltopentaose | 15.3 | 55.0 |
| Maltohexaose | 10.1 | 58.1 |
| Maltoheptaose | 8.5 | 63.6 |
| Amylose | 4.0 | 64.9 |
| Partial starch | 3.8 | 62.7 |
| hydrolyzate | | |

Note:
The symbol “A” means that after contacting α-isomaltosylglucosaccharide-forming enzyme with each saccharide, isomaltodextranase was further allowed to act on the resulting mixture. The symbol “B” means that α-isomaltosylglucosaccharide-forming enzyme and isomaltodextranase were used in combination.

As evident from the results in Table 21, from every saccharide tested, isomaltose was formed via the action of α-isomaltosylglucosaccharide-forming enzyme and isomaltodextranase. It was revealed that, in the case of sequentially contacting α-isomaltosylglucosaccharide-forming enzyme and isomaltodextranase with each saccharide, the formation yield of isomaltose was relatively low as about 15%, while in the case of contacting these enzymes in a combinative manner with any of the saccharides, the formation yield of isomaltose was improved, particularly, it was improved up to 60% or higher when acted on maltoheptaose, amylose, and partial starch hydrolyzate. The mechanism of forming isomaltose by the combination use of α-isomaltosylglucosaccharide-forming enzyme and isomaltodextranase would be as follows:

(1) α-Isomaltosylglucosaccharide-forming enzyme acts on a glucose residue at the non-reducing end of an α-1,4 glucan chain such as amylose and partial starch hydrolyzate and transfers the glucose residue to the C-6 hydroxyl group of another glucose residue at the non-reducing end of another α-1,4 glucan chain to form a α-1,4 glucan chain having an α-isomaltosyl residue at the non-reducing end;

(2) Isomaltodextranase acts on an α-1,4 glucan chain having an isomaltosyl residue at the non-reducing end and hydrolyzes the α-1,4 linkage between the isomaltosyl residue and hydrolyzes the α-1,4 linkage between the isomaltosyl residue and the α-1,4 glucan chain to form a glucan chain, free of the isomaltose, with a lowered glucose polymerization degree by two; and

(3) The released α-1,4 glucan chain is again sequentially received the steps (1) and (2) to newly form isomaltose.

It would be estimated that, through the combination use of α-isomaltosylglucosaccharide-forming enzyme and isomaltodextranase, the formation yield of isomaltose would be increased by the repeated action of the enzymes on α-1,4 glucan chains as described above.

Experiment 18

### Effect of the Addition of Isoamylase

Aqueous solutions of “PINE-DEX #100”, a partial starch hydrolyzate, having a final concentration of 5% and 1 mM calcium chloride, were prepared, admixed with 0.2 unit/g starch of the purified α-isomaltosylglucosaccharide-forming enzyme from Strain C11 in Experiment 4-1, 100 units/g starch of the isomaltodextranase in Experiment 15, and O-250 units/g starch of an isoamylase specimen from a microorganism of the species Pseudomonas amyloderamosa commercialized by Hayashibara Biochemical Laboratories, Inc., Okayama, Japan, incubated at 40° C. and pH 5.5 for 65 hours, and then heated at 100° C. for 15 min to inactivate the enzymes used. The formed isomaltose was quantified on HPLC. The results are in Table 22.

TABLE 22

| Amount of isoamylase added | Formation yield of |
|---|---|
| (unit) | isomaltose (%) |
| 0 | 62.7 |
| 50 | 65.1 |
| 250 | 71.1 |

As evident form the results in Table 22, it was revealed that the addition of isoamylase increases the formation yield of isomaltose.

Experiment 19

### Influence of the Concentration of Partial Starch Hydrolyzate

Eight types of aqueous solutions with different concentrations of “PINE-DEX #100”, a partial starch hydrolyzate with a DE of about 2-5, having final concentrations of 1-40% and 1 mM calcium chloride, were prepared. To each aqueous solution 0.2 unit/g starch of the purified α-isomaltosylglucosaccharide-forming enzyme from Strain C11 in Experiment 4-1, 100 units/g starch of the isomaltodextranase in Experiment 15, and 250 units/g starch of an isoamylase specimen from a microorganism of the species Pseudomonas amyloderamosa commercialized by Hayashibara Biochemical Laboratories, Inc., Okayama, Japan, incubated at 40° C. and pH 5.5 for 65 hours, and then heated at 100° C. for 15 min to inactivate the enzymes used. The formed isomaltose was quantified on HPLC. The results are in Table 23.

TABLE 23

| Concentration of | Formation yield of |
|---|---|
| PINE-DEX (%) | isomaltose (%) |
| 1 | 73.0 |
| 2.5 | 72.8 |
| 5 | 71.1 |
| 10 | 67.0 |
| 15 | 63.7 |
| 20 | 60.7 |
| 30 | 55.4 |
| 40 | 50.7 |

As evident from the results in Table 23, it was revealed that the formation of yield of isomaltose was about 73% at a concentration of one percent of partial starch hydrolyzate, while it was about 51% at a relatively high concentration of 40%. Thus, the formation of yield of isomaltose changes depending on the concentration of partial starch hydrolyzate as a substrate.

Experiment 20

### Influence of the Liquefaction Degree of Starch

Corn starch was prepared into a 15% starch suspension which was then mixed with 0.1% calcium carbonate, adjusted to pH 6.0, admixed with 0.2-2.0% per gram starch of “TERMAMYL 6OL”, an α-amylase specimen commercialized by Novo Indutri A/S, Copenhagen, Denmark, allowed to react at 95° C. for 10 min, and autoclaved at 120° C. Thereafter, the reaction mixture was promptly cooled to about 40° C. to obtain a liquefied solution with a DE of 3.2-20.5 which was then adjusted to pH 5.5, admixed with 0.2 unit/g starch of the purified α-isomaltosylglucosaccharide-forming enzyme from Strain C11 in Experiment 4-1, 100 units/g starch of the isomaltodextranase in Experiment 15, and 250 units/g starch of an isoamylase specimen from a microorganism of the species Pseudomonas amyloderamosa commercialized by Hayashibara Biochemical Laboratories, Inc., Okayama, Japan, incubated at 40° C. for 65 hours, and then heated at 100° C. for 15 min to inactivate the enzymes used. The formed isomaltose was quantified on HPLC. The results are in Table 24.

TABLE 24

| Amount of α-amylase | | |
|---|---|---|
| used (% (w/w) | | Formation yield of |
| per gram starch) | DE | isomaltose (%) |
| 0.2 | 3.2 | 71.5 |
| 0.4 | 4.8 | 71.0 |
| 0.6 | 7.8 | 66.2 |
| 1.0 | 12.5 | 59.8 |
| 1.5 | 17.3 | 53.2 |
| 2.0 | 20.5 | 47.9 |

As evident from the results in Table 24, it was revealed that the liquefaction degree of starch influences the formation yield of isomaltose using α-isomaltosylglucosaccharide-forming enzyme and isomaltodextranase; the lower the liquefaction degree or the lower the DE, the higher the formation yield of isomaltose becomes, in reverse, the higher the liquefaction degree or the higher the DE, the lower the formation yield of isomaltose becomes; it was revealed that the liquefaction degree should preferably be a DE not higher than 20, preferably, DE not higher than 12, more preferably, DE not higher than five.

Experiment 23

### Effect of the Addition of Cyclodextrin Glucanotransferase and Glucoamylase

Aqueous solutions of “PINE-DEX #100”, a partial starch hydrolyzate, having a final concentration of 20% and 1 mM calcium chloride, were prepared and admixed with 0.2 unit/g starch of the purified α-isomaltosylglucosaccharide-forming enzyme from Strain C11 in Experiment 4-1, 100 units/g starch of the isomaltodextranase in Experiment 15, 250 units/g starch of an isoamylase specimen from a microorganism of the species Pseudomonas amyloderamosa commercialized by Hayashibara Biochemical Laboratories, Inc., Okayama, Japan, and O-0.5 unit/g starch of a CGTase specimen from a microorganism of the species Bacillus stearothermophilus, commercialized by Hayashibara Biochemical Laboratories, Inc., Okayama, Japan, and incubated with these enzymes at 40° C. and pH 5.5 for 65 hours. Thereafter, each reaction mixture was heated at 100° C. for 15 min to inactivate the enzymes, admixed with 20 units/g starch of “XL-4”, a glucoamylase commercialized by Nagase Biochemicals, Ltd., Kyoto, Japan, incubated at 50° C. for 24 hours, and heated at 100° C. for 20 min to inactivate the remaining enzyme. The formed isomaltose was quantified on HPLC. The results are in Table 25.

TABLE 25

| Amount of CGTase added | Formation yield of |
|---|---|
| (unit per gram starch) | isomaltose (%) |
| 0 | 60.7 |
| 0.1 | 62.9 |
| 0.25 | 65.0 |
| 0.5 | 66.4 |

As evident from the results in Table 25, it was revealed that the addition of CGTase to the enzymatic system of α-isomaltosylglucosaccharide-forming enzyme and isomaltodextranase increases the formation yield of isomaltose. The object of using the glucoamylase was to increase the formation of isomaltose by releasing D-glucose residue(s) from a saccharide composed of isomaltose and at least one D-glucose residue.

With reference to the following Examples A and B, the process for producing isomaltose or high isomaltose content products according to the present invention and uses thereof are disclosed in detail:

### EXAMPLE A-1

About 100 L of an aqueous solution of phytoglycogen from corn, commercialized by Q.P. Corporation, Tokyo, Japan, were adjusted to give a concentration of 4% (w/v), pH 6.0, and a temperature of 30° C., admixed with one unit/g starch of a purified α-isomaltosylglucosaccharide-forming enzyme from Strain C11 obtained by the method in Experiment 4-1, and 10 units/g starch of an α-isomaltosyl-transferring enzyme from Strain C11 obtained by the method in Experiment 4-4, allowed to react for 48 hours, and heated at 100° C. for 10 min to inactivate the remaining enzymes. The resulting mixture was sampled and quantified the formation yield of cyclotetrasaccharide on HPLC to be about 84% with respect to the saccharide composition, wherein HPLC was carried out under the conditions of: Column, “SHODEX KS-801 COLUMN” comercialized by Showa Denko K. K., Tokyo, Japan; 60° C., an inner column temperature; 0.5 ml/min, a flow rate of water as an eluent; and detection by “RI-8012”, a diffraction refractometer commercialized by Tosoh Corporation, Tokyo, Japan. After adjusted to pH 5.0 and 45° C., the resulting reaction mixture was admixed with 1,500 units/g starch “TRANSGLUCOSIDASE L AMANO™”, an α-glucosidase commercialized by Amano Pharmaceutical Co., Ltd., Aichi, Japan, and 75 units/g starch of “XL-4”, a glucoamylase specimen commercialized by Nagase Biochemicals, Ltd., Kyoto, Japan, to hydrolyze the remaining reducing-oligosaccharides. Then, the resulting mixture was adjusted to give a pH 5.8 by the addition of sodium hydroxide, kept at 90° C. for one hour to inactivate the remaining enzymes, and filtered to remove insoluble substances. The filtrate was concentrated using “HOLLOSEP® HR5155PI”, a reverse osmosis membrane commercialized by Toyobo Co., Ltd., Tokyo, Japan, up to give a concentration of about 16% (w/v). Then, the concentrate was in a usual manner decolored, desalted, filtered, and concentrated into about 6.2 kg of a saccharide solution having about 3,700 g of solid contents. The saccharide solution was fed to a column packed with about 225 L of “AMBERLITE CR-1310 (Na⁺-form)”, a strong-acid cation-exchanger commercialized by Japan Organo Co., Ltd., Tokyo, Japan, and chromatographed at a column temperature of 60° C. and a flow rate of about 45 L/h. While monitoring the saccharide composition of the eluate by the above HPLC, fractions of cyclotetrasaccharide with a purity of 98% or higher were collected and pooled, and then in a usual manner, desalted, decolored, filtered, and concentrated to obtain about 7.5 kg saccharide solution with a solid content of about 2,500 g. The HPLC revealed that the saccharide solution had a purity of about 99.5% of cyclotetrasaccharide. The obtained saccharide solution containing cyclotetrasaccharide was concentrated by an evaporator to give a concentration of about 50%, and about 5 kg of the concentrate was placed in a cylindrical plastic container and cooled from 65° C. to 20° C. over about 20 hours under gentle stirring conditions to crystallize cyclotetrasaccharide. Thereafter, the resulting massecuite was centrifuged to separate 1,360 g of cyclotetrasaccharide crystal on a wet weight, which was then dried at 60° C. for three hours to obtain 1,170 g of a powdery cyclotetrasaccharide crystal. The powdery crystal was analyzed for saccharide composition on HPLC to reveal that it had a quite high purity of at least 99.9% of cyclotetrasaccharide.

The powdery cyclotetrasaccharide crystal thus obtained was dissolved in deionized water to give a concentration of one percent, pH 5.5, and 50° C., followed by admixing with 500 units/g solids of an isomaltodextranase specimen obtained by the method in Experiment 15, and incubating the mixture at pH 5.5 and 50° C. for 70 hours. After completion of the enzymatic reaction, the reaction mixture was heated to 95° C. and kept at the temperature for 10 min, cooled, and filtered. The resulting filtrate was in a usual manner decolored with an activated charcoal, desalted and purified using ion-exchange resins in H— and OH-forms, and further concentrated to give a concentration of 75%. Thus, a high isomaltose content syrup was obtained in a yield of about 95%, d.s.b.

The product contained 96.1% isomaltose, 2.8% ring-opened tetrasaccharide, and 1.1% other saccharides, d.s.b. Since the product substantially free of crystallization has a satisfactory humectancy, low-sweetness, osmosis controllability, filler-imparting ability, gloss-imparting ability, viscosity, ability of preventing crystallization of other saccharides, insubstantial fermentability, ability of preventing retrogradation of starches, etc., it can be arbitrarily used in foods, beverages, health foods, feeds, pet foods, cosmetics, pharmaceuticals, tobaccos, and cigarettes.

### EXAMPLE A-2

A high isomaltose content syrup, obtained by the method in Example A-1, was subjected to column chromatography using “AMBERLITE CR-1310 (Na⁺-form)”, a strong-acid cation exchanger commercialized by Japan Organo Co., Ltd., Tokyo, Japan. The resin was packed into 10 jacketed stainless steel columns having a diameter of 12.5 cm, which were then cascaded in series to give a total gel bed depth of 16 m. Under the conditions of keeping the inner column temperature at 40° C., the above saccharide syrup was fed to the columns in a volume of 1.5% (v/v) and fractionated by feeding to the columns hot water heated to 40° C. at an SV (space velocity) of 0.2 to obtain high isomaltose content fractions while monitoring the saccharide composition of eluate on HPLC. Then, the fractions were pooled and purified to obtain a high isomaltose content solution in a yield of about 80%, d.s.b. The solution was in a usual manner decolored; desalted, and concentrated into an about 75%, d.s.b., of high isomaltose content syrup.

The product contained a high purity isomaltose with a purity of at least 99.9%, d.s.b. Since the product substantially free of crystallization has a satisfactory humectancy, low-sweetness, osmosis controllability, filler-imparting ability, gloss-imparting ability, viscosity, ability of preventing crystallization of other saccharides, insubstantial fermentability, ability of preventing retrogradation of starches, etc., it can be arbitrarily used in foods, beverages, health foods, feeds, pet foods, cosmetics, pharmaceuticals, tobaccos, and cigarettes.

### EXAMPLE A-3

A tapioca starch was prepared into an about 20% starch suspension, admixed with calcium carbonate to give a concentration of 0.1%, adjusted to pH 6.5, further admixed with 0.3% per gram starch, d.s.b., of “TERMAMYL 60L”, an α-amylase commercialized by Novo Industri A/S, Copenhagen, Denmark, and then heated at 95° C. for about 15 min. Thereafter, the mixture was autoclaved at 120° C. for 20 min and then promptly cooled to about 40° C. to obtain a liquefied solution with a DE of about four. To the liquefied solution was added 0.2 unit/g starch of an α-isomaltosylglucosaccharide-forming enzyme obtained by the method in Experiment 2-1, 100 units/g starch of an isomaltodextranase obtained by the method in Experiment 15, 250 units/g starch of an isomaltodextranase specimen commercialized by Hayashibara Biochemical Laboratories, Inc., Okayama, Japan, and 0.5 unit/g starch of a CGTase specimen commercialized by Hayashibara Biochemical Laboratories, Inc., Okayama, Japan, and then enzymatically reacted at pH 5.5 and 40° C. for 64 hours. The reaction mixture was kept at 95° C. for 30 min, adjusted to 50° C., 10 units/g solids of “GLUCOZYME”, a glucoamylase preparation commercialized by Nagase Biochemicals, Ltd., Kyoto, Japan, and then enzymatically reacted for 24 hours. The reaction mixture thus obtained was heated to and kept at 95° C. for 30 min, and then cooled and filtered. The filtrate was in a conventional manner decolored with an activated charcoal, desalted and purified with ion exchangers in H— and OH-forms, and then concentrated, dried, pulverized, and granulated into isomaltose granules in a yield of about 95%, d.s.b.

The product contains, on a dry solid basis, 11.0% glucose, 66.5% isomaltose, 2.4% other disaccharides, and 20.1% trisaccharides or higher. Since the product has a satisfactory humectancy, low-sweetness, osmosis controllability, filler-imparting ability, gloss-imparting ability, viscosity, ability of preventing crystallization of other saccharides, insubstantial fermentability, ability of preventing retrogradation of starches, etc., it can be arbitrarily used in foods, beverages, health foods, feeds, pet foods, cosmetics, pharmaceuticals, tobaccos, and cigarettes.

### EXAMPLE A-4

Bacillus globisporus C9 strain, FERM BP-7143, was cultured by a fermentor for 48 hours in accordance with the method in Experiment 1. After completion of the culture, the resulting culture was filtered with an SF membrane to remove cells and to collect about 18 L of a culture supernatant. Then the culture supernatant was concentrated with a UF membrane to collect about one liter of a concentrated enzyme solution containing 8.8 units/ml of an α-isomaltosylglucosaccharide-forming enzyme and 26.7 units/ml of an α-isomaltosyl-transferring enzyme. A potato starch was prepared into an about 27% starch suspension which was then admixed with 0.1% calcium carbonate, adjusted to pH 6.5, admixed with 0.3% per gram starch, d.s.b., of “TERMAMYL 60L”, an α-amylase commercialized by Novo Industri A/S, Copenhagen, Denmark, and then sequentially heated at 95° C. for 15 min, autoclaved at 120° C. for 20 min, and promptly cooled to about 40° C. to obtain a liquefied solution with a DE of about four. To the liquefied solution were added 0.25 ml per gram of starch of the above concentrated enzyme solution containing the α-isomaltosylglucosaccharide-forming enzyme and the α-isomaltosyl-transferring enzyme, 100 units/g starch of an isomaltodextranase obtained by the method in Experiment 15, 250 units/g starch of an isoamylase specimen commercialized by Hayashibara Biochemical Laboratories, Inc., Okayama, Japan, and 0.5 unit/g starch of a CGTase specimen commercialized by Hayashibara Biochemical Laboratories, Inc., Okayama, Japan, and then the resulting mixture was subjected to enzymatic reaction at pH 5.5 and 40° C. for 70 hours. The reaction mixture was heated to and kept at 95° C. for 10 min, adjusted to 50° C., admixed with 20 units/g starch of “GLUCOZYME”, a glucoamylase preparation commercialized by Nagase Biochemicals, Ltd., Kyoto, Japan, and then enzymatically reacted for 24 hours. The reaction mixture thus obtained was heated to and kept at 95° C. for 30 min, and then cooled and filtered. The filtrate was in a conventional manner decolored with an activated charcoal, desalted and purified with ion exchangers in H— and OH-forms and concentrated to obtain a 75% high isomaltose content syrup in a yield of about 95%, d.s.b.

The product contains, on a dry solid basis, 32.6% glucose, 59.4% isomaltose, 1.2% other disaccharides, and 6.8% trisaccharides or higher. Since the product substantially free of crystallization has a satisfactory humectancy, low-sweetness, osmosis controllability, filler-imparting ability, gloss-imparting ability, viscosity, ability of preventing crystallization of other saccharides, insubstantial fermentability, ability of preventing retrogradation of starches, etc., it can be arbitrarily used in foods, beverages, health foods, feeds, pet foods, cosmetics, pharmaceuticals, tobaccos, and cigarettes.

### EXAMPLE A-5

To increase the isomaltose content in the high isomaltose content syrup in Example A-4 as a material saccharide solution, in accordance with the method in Example A-2, the syrup was subjected to column chromatography using a strong-acid cation exchange resin, followed by collecting the resulting high isomaltose content fractions which were then pooled and concentrated to obtain a high isomaltose content syrup in a yield of about 60%, d.s.b.

The product contains, on a dry solid basis, 4.8% glucose, 85.3% isomaltose, 3.9% other disaccharides, and 6.0% trisaccharides or higher. Since the product substantially free of crystallization has a satisfactory humectancy, low-sweetness, osmosis controllability, filler-imparting ability, gloss-imparting ability, viscosity, ability of preventing crystallization of other saccharides, insubstantial fermentability, ability of preventing retrogradation of starches, etc., it can be arbitrarily used in foods, beverages, health foods, feeds, pet foods, cosmetics, pharmaceuticals, tobaccos, and cigarettes.

### EXAMPLE B-1

Sweetener

To 0.8 part by weight of a high isomaltose content powder, obtained by the method in Example A-1, were homogeneously added 0.2 part by weight of “TREHA®”, a crystalline trehalose hydrate commercialized by Hayashibara Shoji Inc., Okayama, Japan, 0.01 part by weight of ”αG SWEET™”, (α-glycosyl stevioside commercialized by Toyo Sugar Refining Co., Tokyo, Japan), and 0.01 part by weight of “ASPARTAME” (L-aspartyl-L-phenylalanine methyl ester). The resulting mixture was fed to a granulator to obtain a granular sweetener. The product has a satisfactory sweetness and about 2-fold higher sweetening power of sucrose. The product is a low-sweetener composition containing isomaltose which is substantially free of crystallization and has satisfactory humectancy and low-sweetness. The product has a satisfactory stability with lesser fear of causing quality deterioration even when stored at ambient temperature.

### EXAMPLE B-2

Hard Candy

One hundred parts by weight of a 55% sucrose solution was admixed while heating with 50 parts by weight of a high isomaltose content syrup obtained by the method in Example A-2. The mixture was then concentrated by heating under reduced pressure to give a moisture content of less than 2%, and the concentrate was mixed with 0.6 part by weight of citric acid and adequate amounts of a lemon flavor and a color, followed by forming in a usual manner the resultant mixture into the desired product. The product is a stable, high quality hard candy which has a satisfactory mouth feel, taste, and flavor, less adsorbs moisture, and does not cause crystallization of sucrose.

### EXAMPLE B-3

Chewing Gum

Three parts by weight of a gum base were melted by heating to an extent to be softened and then admixed with two parts by weight of anhydrous crystalline maltitol anhydride, two parts by weight of xylitol, two parts by weight of a high isomaltose content syrup obtained by the method in Example A-5, and one part by weight of trehalose, and further mixed with adequate amounts of a flavor and a color. The mixture was in a usual manner kneaded by a roll and then shaped and packed to obtain the desired product. The product was a relatively low cariogenic and caloric chewing gum having a satisfactory texture, taste, and flavor.

### EXAMPLE B-4

Powdery Peptide

One part by weight of 40% of “HINUTE S”, a peptide solution of edible soy beans commercialized by Fuji Oil Co., Ltd., Tokyo, Japan, was mixed with two parts by weight of a high isomaltose content syrup obtained by the method in Example A-4, and the resultant mixture was placed in a plastic vat, dried in vacuo at 50° C., and pulverized to obtain a powdery peptide. The product, having a satisfactory flavor and taste, can be arbitrary used as a material for low-caloric confectioneries such as premixes, sherbets and ice creams, as well as a material for controlling intestinal conditions, health food, and substantially non-digestible edible fibers used for fluid diets for oral administration and intubation feeding.

### EXAMPLE B-5

Bath Salt

One part by weight of a peel juice of “yuzu” (a Chinese lemon) was mixed with 10 parts by weight of a high isomaltose content syrup obtained by the method in Example A-3, and one part by weight of cyclotetrasaccharide, and the mixture was pulverized into an isomaltose powder containing a peel juice of yuzu.

A bath salt was obtained by mixing five parts by weight of the above powder with 90 parts by weight of grilled salt, two parts by weight of crystalline trehalose hydrate, one part by weight of silicic anhydride, and 0.5 part by weight of “αG HESPERIDIN”, α-glucosyl hesperidin commercialized by Hayashibara Shoji, Inc., Okayama, Japan.

The product is a high quality bath salt enriched with yuzu flavor and used by diluting in hot bath water by 100-10,000 folds, and it moisturizes and smooths the skin and does not make you feel cold after taking a bath therewith.

### EXAMPLE B-6

### Cosmetic Cream

Two parts by weight of polyoxyethylene glycol monostearate, five parts by weight of glyceryl monostearate, self-emulsifying, two parts by weight of a high isomaltose content syrup obtained by the method in Example A-2, one part by weight of “αG RUTIN”, α-glucosyl rutin commercialized by Hayashibara Shoji, Inc., Okayama, Japan, one part by weight of liquid petrolatum, 10 parts by weight of glyceryl tri-2-ethylhexanoate, and an adequate amount of an antiseptic were dissolved by heating in a usual manner. The resulting solution was admixed with two parts by weight of L-lactic acid, five parts by weight of 1,3-butylene glycol, and 66 parts by weight of refined water, followed by emulsifying the mixture with a homogenizer and further admixing by stirring with an adequate amount of a flavor stirring to obtain a cosmetic cream. The product has an antioxidant activity and a relatively high stability, and these render it advantageously useful as a high quality sunscreen, skin-refining agent, and skin-whitening agent.

### EXAMPLE B-7

Toothpaste

A toothpaste was obtained by mixing 45 parts by weight of calcium secondary phosphate, 1.5 parts by weight of sodium lauryl sulfate, 25 parts by weight of glycerine, 0.5 part by weight of polyoxyethylene sorbitan laurate, 15 parts by weight of a high isomaltose content syrup obtained by the method in Example A-5, 0.02 part by weight of saccharine, 0.05 part by weight of an antiseptic, and 13 parts by weight of water. The product has an improved after taste and a satisfactory feeling after use without deteriorating the washing power of the surfactant.

### EXAMPLE B-8

Solid Preparation for Fluid Diet

One hundred parts by weight of a high isomaltose content syrup obtained by the method in Example A-1, 200 parts by weight of crystalline trehalose hydrate, 200 parts by weight of high maltotetraose content powder, 270 parts by weight of an egg yolk powder, 209 parts by weight of a skim milk powder, 4.4 parts by weight of sodium chloride, 1.8 parts by weight of potassium chloride, four parts by weight of magnesium sulfate, 0.01 part by weight of thiamine, 0.1 part by weight of sodium L-ascorbate, 0.6 part by weight of vitamin E acetate, and 0.04 part by weight of nicotinamide were mixed. Twenty-five grams aliquots of the resulting composition were injected into moisture-proof laminated small bags which were then heat sealed to obtain the desired product.

The product is a fluid diet that has a satisfactory intestinal-controlling action. One bag of the product is dissolved in about 150-300 ml of water into a fluid diet and arbitrarily used by administering orally or intubationally into nasal cavity, stomach, intestines, etc., to supplement energy to living bodies.

### EXAMPLE B-9

Tablet

To 50 parts by weight of aspirin were sufficiently admixed with 14 parts by weight of a high isomaltose content syrup obtained by the method in Example A-2, and four parts by weight of corn starch. The resulting mixture was in a usual manner tabletted by a tabletting machine to obtain a tablet, 680 mg each, 5.25 mm in thickness.

The tablet, processed using the filler-imparting ability of isomaltose, has substantially no hygroscopicity, a sufficient physical strength and a quite satisfactory degradability in water.

### EXAMPLE B-10

Sugar Coated Tablet

A crude tablet as a core, 150 mg weight, was sugar coated with a first solution consisting of 40 parts by weight of a high isomaltose content syrup obtained by the method in Example A-1, two parts by weight of pullulan having an average molecular weight of 200,000, 30 parts by weight of water, 25 parts by weight of talc, and three parts by weight of titanium oxide until the total weight reached about 230 mg. The resultant was then sugar coated with a second solution consisting of 65 parts by weight of crystalline cyclotetrasaccharide, one part by weight of pullulan, and 34 parts by weight of water, and glossed with a liquid wax to obtain a sugar coated tablet having a satisfactory gloss and appearance. The product has a relatively high shock tolerance and retains its high quality for a relatively-long period of time.

### EXAMPLE B-11

Ointment for Treating Trauma

To 100 parts by weight of a high isomaltose content syrup obtained by the method in Example A-5 and 300 parts by weight of maltose was added 50 parts by weight of methanol dissolving three parts by weight of iodine. The resulting mixture was admixed with 200 parts by weight of a 10% (w/v) aqueous pullulan solution to obtain the captioned product with an adequate extensibility and adhesiveness. The product is a high-valued ointment in which the dispersion of iodine and methanol is well inhibited by isomaltose and which is relatively low in change during storage.

Because the product exerts a sterilizing action by iodine and acts, based on maltose, as an energy-supplementing agent to living cells, it shortens the curing term and well cures the affected parts and surfaces.

### Industrial Applicability

As described above, the present invention relates to a novel process for producing isomaltose and uses thereof, more particularly, to a process for producing isomaltose characterized in that it comprises the steps of allowing α-isomaltosylglucosaccharide-forming enzyme, in the presence or the absence of α-isomaltosyl-transferring enzyme, to act on saccharides having both a glucose polymerization degree of at least two and α-1,4 glucosidic linkage as a linkage at the non-reducing end to form α-isomaltosylglucosaccharides, which have a glucose polymerization degree of at least three, α-1,6 glucosidic linkage as a linkage at the non-reducing end, and α-1,4 glucosidic linkage as a linkage other than the non-reducing end, and/or to form cyclo{→6)-α-D-glucopyranosyl-(1→3)-α-D-glucopyranosyl-(1→6)-α-D-glucopyranosyl-(1→3)-α-D-glucopyranosyl-(1→}; allowing isomaltose-releasing enzyme to act on the formed saccharides to release isomaltose; and collecting the released isomaltose; and relates to uses thereof. The isomaltose and high isomaltose content products of the present invention do not substantially crystallize and have useful properties of humectancy, low-sweetness, osmosis-controlling ability, filler-imparting ability, gloss-imparting ability, viscosity, crystallization-preventing ability for saccharides, insubstantial fermentability, retrogradation-preventing ability for gelatinized starches, etc. Thus, the isomaltose and high isomaltose content products can be arbitrarily used in foods, beverages, health foods, feeds, pet foods, cosmetics, pharmaceuticals, tobaccos, and cigarettes.

The present invention having these outstanding effects and functions is a significant invention that will greatly contribute to this art.

1. A process for producing isomaltose, characterized in that it comprises the steps of:
allowing α-isomaltosylglucosaccharide-forming enzyme, in the presence or the absence of α-isomaltosyl-transferring enzyme, to act on a saccharide having both a glucose polymerization degree of at least two and α-1,4 glucosidic linkage as a linkage at the non-reducing end to form α-isomaltosylglucosaccharides which have a glucose polymerization degree of at least three, α-1,6 glucosidic linkage as a linkage at the non-reducing end, and α-1,4 glucosidic linkage as a linkage other than the non-reducing end, and/or to form cyclo{→6)-α-D-glucopyranosyl-(1→3)-α-D-glucopyranosyl-(1→6)-α-D-glucopyranosyl-(1→3)-α-D-glucopyranosyl-(1→};
allowing isomaltose-releasing enzyme to act on the formed saccharide(s) to release isomaltose; and
collecting the released isomaltose.

2. The process of claim 1, wherein in the step of allowing α-isomaltosylglucosaccharide-forming enzyme to act on the saccharide, one or more enzymes selected from the group consisting of α-isomaltosyl-transferring enzyme, cyclomaltodextrin glucanotransferase, α-glucosidase, glucoamylase, and starch debranching enzyme are allowed to act on the saccharide.

3. The process of claim 1, wherein after the step of allowing α-isomaltosylglucosaccharide-forming enzyme to act on the saccharide, one or more enzymes selected from the group consisting of α-isomaltosyl-transferring enzyme, cyclomaltodextrin glucanotransferase, α-glucosidase, glucoamylase, and starch debranching enzyme are allowed to act on the resulting mixture in the above step.

4. The process of claim 1, 2 or 3, wherein said saccharide having both a glucose polymerization degree of at least two and α-1,4 glucosidic linkage as a linkage at the non-reducing end is one selected from the group consisting of maltooligosaccharides, maltodextrins, amylodextrins, amyloses, amylopectins, soluble starches, liquefied starches, and glycogens.

5. A process for producing isomaltose, characterized in that it comprises the step of:
allowing isomaltose-releasing enzyme to act on a saccharide mixture comprising at least two saccharides selected from the group consisting of cyclo{→6)-α-D-glucopyranosyl-(1→3)-α-D-glucopyranosyl-(1→6)-α-D-glucopyranosyl-(1→3)-α-D-glucopyranosyl-(1→},α-glucosyl-(1→6)-α-glucosyl-(1→3)-α-glucosyl-(1→6)-α-glucose, and panose to release isomaltose; and
collecting the released isomaltose.

6. The process of claim 5, wherein said cyclo{→6)-α-D-glucopyranosyl-(1→3)-α-D-glucopyranosyl-(1→6)-α-D-glucopyranosyl-(1→3)-α-D-glucopyranosyl-(1→},α-glucosyl-(1→6)-α-glucosyl-(1→3)-α-glucosyl-(1→6)-α-glucose, and panose are prepared by allowing an enzyme to act on a saccharide having both a glucose polymerization degree of at least two and α-1,4 glucosidic linkage as a linkage at the non-reducing end.

7. The process of any one of claims 1 to 6, wherein said α-isomaltosyl-transferring enzyme has the following physicochemical properties:
(1) Action
Acting on a saccharide, which has a glucose polymerization degree of at least three, α-1,6 glucosidic linkage as a linkage at the non-reducing end, and α-1,4 glucosidic linkage as a linkage other than the non-reducing end, to form cyclotetrasaccharide having a structure of cyclo{→6)-α-D-glucopyranosyl-(1→3)-α-D-glucopyranosyl-(1→6)-α-D-glucopyranosyl-(1→3)-α-D-glucopyranosyl-(1→} through α-isomaltosyl transfer;
(2) Molecular weight
Having a molecular weight of about 82,000 to about 132,000 daltons when determined on SDS-PAGE;
(3) Isoelectric point (pI)
Having an isoelectric point of about 5.0 to about 6.1 when determined on isoelectrophoresis using ampholine;
(4) Optimum temperature
Having an optimum temperature of about 45° C. to about 50° C. when incubated at a pH of 6.0 for 30 min;
(5) Optimum pH
Having an optimum pH of about 5.5 to about 6.0 when incubated at 35° C. for 30 min;
(6) Thermal stability
Stable up to a temperature of about 40°0 C. when incubated at a pH of 6.0 for 60 min; and
(7) pH Stability
Stable at a pH of about 4.0 to about 9.0 when incubated at 4° C. for 24 hours.

8. The process of any one of claims 1 to 6, wherein said α-isomaltosylglucosaccharide-forming enzyme having the following physicochemical properties:
(1) Action
Forming a saccharide, which has a glucose polymerization degree of at least three, α-1,6 glucosidic linkage as a linkage at the non-reducing end, and α-1,4 glucosidic linkage other than the linkage at the non-reducing end, by catalyzing the α-glucosyl-transferring reaction from a saccharide having both a glucose polymerization degree of at least two and having α-1,4 glucosidic linkage as a linkage at the non-reducing end without substantially increasing the reducing power;
(2) Molecular weight
Having a molecular weight of about 117,000 to about 160,000 daltons when determined on SDS-PAGE;
(3) Isoelectric point (pI)
Having an isoelectric point of about 4.7 to about 5.7 when determined on isoelectrophoresis using ampholine;
(4) Optimum temperature
Having an optimum temperature of about 40° C. to about 45° C. when incubated at a pH of 6.0 for 60 min, or an optimum temperature of about 45° C. to about 50° C. when incubated in the presence of 1 mM Ca²⁺;
(5) Optimum pH
Having an optimum pH of about 6.0 to about 6.5 when incubated at 35° C. for 60 min;
(6) Thermal stability
Stable up to a temperature of about 35° C. to 40° C., or a temperature of about 40° C. to about 45° C. in the presence of 1 mM Ca²⁺; and
(7) pH Stability
Stable at a pH of about 4.5 to about 10.0 when incubated at 4° C. for 24 hours.

9. The process of any one of claims 1 to 8, wherein in the step of collecting the released isomaltose, a column chromatography using an alkaline metal and/or alkaline earth metal strong-acid cation exchange resin is used.

10. The process of any one of claims 1 to 9, wherein the collected isomaltose is a high isomaltose content syrup having an isomaltose content of at least 40% (w/w), on a dry solid basis.

11. A high isomaltose content syrup obtained by the process of any one of claims 1 to 10, characterized in that it comprises, on a dry solid basis, 40-99% (w/w) of isomaltose and 1-60% (w/w) of one or more saccharides selected from the group consisting of glucose, maltose, maltotriose, maltotetraose, starch hydrolyzates, α-isomaltosylglucosaccharides, and α-glucosyl-(1→6)-α-glucosyl-(1→3)-α-glucosyl-(1→6)-α-glucose.

12. A food product or health food, comprising the isomaltose obtained by the method of any one of claims 1 to 10, or the high isomaltose content product of claim 11.

13. A feed or pet food, comprising the isomaltose obtained by the method of any one of claims 1 to 10, or the high isomaltose content product of claim 11.

14. A cosmetic comprising the isomaltose obtained by the method of any one of claims 1 to 10, or the high isomaltose content product of claim 11.

15. A pharmaceutical comprising the isomaltose obtained by the method of any one of claims 1 to 10, or the high isomaltose content product of claim 11.

16. A process for producing a food product or health food, characterized in that it comprises a step of using the isomaltose obtained by the method of any one of claims 1 to 10, or the high isomaltose content product of claim 11.

17. A process for producing a feed or pet food, characterized in that it comprises a step of using the isomaltose obtained by the method of any one of claims 1 to 10, or the high isomaltose content product of claim 11.

18. A process for producing a cosmetic, characterized in that it comprises a step of using the isomaltose obtained by the method of any one of claims 1 to 10, or the high isomaltose content product of claim 11.

19. A process for producing a pharmaceutical, characterized in that it comprises a step of using the isomaltose obtained by the method of any one of claims 1 to 10, or the high isomaltose content product of claim 11.
